(19) United States
(12) Patent Application Publication
(10) Pub. No.: US 2025/0312413 A1
(43) Pub. Date: Oct. 9, 2025

(54) HUMANIZED RECOMBINANT VACCINIA VIRUS COMPLEMENT CONTROL PROTEIN (hrVCP) FOR PROTECTION OF COGNITIVE FUNCTION

(71) Applicant: InFlaMed Inc., Louisville, KY (US)

(72) Inventors: Girish J. Kotwal, Louisville, KY (US); Amod P. Kulkarni, Louisville, KY (US)

(21) Appl. No.: 19/025,559

(22) Filed: Jan. 16, 2025

Related U.S. Application Data
(60) Provisional application No. 63/575,141, filed on Apr. 5, 2024.

(51) Int. Cl. A61K 38/16; A61K 9/00; A61P 25/28; C12N 7/00

(52) U.S. Cl. CPC A61K 38/162; A61K 9/0043; A61P 25/28; C12N 7/00; C12N 2710/24032

(57) ABSTRACT

A method of reducing an APOE ε4 mediated cognitive deficit is provided and comprises administering to a subject in need thereof an effective amount of a recombinant vaccinia virus complement control protein (hrVCP) polypeptide having a modified amino acid sequence including one or more amino acid substitutions to an amino acid sequence as set forth in SEQ ID NO: 2, wherein the one or more amino acid substitutions are selected from the group consisting of H98Y, E102K, E108K, E120K, and combinations thereof, provided at least one of the substitutions is E102K. Methods of treating dementia characterized by an APOE ε4 mediated cognitive deficit and methods of reducing complement activation mediated by APOE ε4 are further provided an include administration of the hrVCP polypeptide.

### RELATED APPLICATIONS

This application claims priority from U.S. Provisional Application Ser. No. 63/575,141, filed Apr. 5, 2024, the entire disclosure of which is incorporated herein by this reference.

### GOVERNMENT INTEREST

This invention was made with government support under grant number 1R43AG080890-01 awarded by the National Institutes of Health. The government has certain rights in the invention.

### REFERENCE TO AN ELECTRONIC SEQUENCE LISTING

The contents of the electronic sequence listing (sequencelisting.xml; Size: 7,647 bytes; and Date of Creation: Jan. 16, 2025) is herein incorporated by reference in its entirety.

### TECHNICAL FIELD

The presently-disclosed subject matter generally relates to the use of humanized recombinant vaccinia virus complement control protein (hrVCP) for protection of cognitive function. In particular, certain embodiments of the presently-disclosed subject matter relate to an intranasal formulation of hrVCP for protection of cognitive function in subjects who are genetically predisposed to Alzheimer's Disease (AD), vascular and/or other forms of dementia due to being carriers of or having an APOE ε4 positive status.

### BACKGROUND

Alzheimer's disease (AD), vascular dementia, and other forms of dementia are devastating neurodegenerative disorders characterized by progressive cognitive decline. The incidence and prevalence of AD is high in the United States and across the globe. About 6.7 million Americans aged 65 and older were living with AD in 2024. However, besides AD, there are other forms of dementia, such as vascular dementia, that affect a significant portion of the world population including in the United States. This number is expected to grow in the future and of those affected individuals, it has been determined that about 15% to 25% have the APOE ε4 allele, with 2% to 5% carrying two copies (homozygous) of this allele. Indeed, the epidemiological studies, molecular biology, and histopathological studies of AD patients and related statistics suggests that genetic factors such as the APOE ε4 allele play important roles in the predisposition and pathogenesis of AD across all races in the United States, such that APOE ε4 carriers are now generally characterized as being genetically predisposed to AD.

Despite decades of research, there is still currently no cure for AD, and available treatments offer only limited benefits. In particular, there is no treatment option available for the individuals in the age group of less than age 55 or greater than age 65, even if they know their status of APOE ε4 allele. Moreover, recently developed amyloid-targeting therapies for AD have been associated with serious adverse events, particularly micro-hemorrhage, and especially in individuals carrying the APOE ε4 allele. The therapies also cannot be used for vascular and other forms of dementia where amyloid and tau are not evident as primary markers.

These current U.S. Food and Drug Administration (FDA) approved treatments targeting the amyloid plaques in patients with early cognitive deficit have had minimal short lived mental health benefits while being accompanied with adverse debilitating effects such as swelling of the brain. In this regard, neuroinflammation has been increasingly recognized as a key driver of AD pathogenesis with the complement system, a part of the innate immune system, playing a central role in mediating neuroinflammation. In fact, the activation of the complement system often leads to a cascade of events that contribute to neuronal damage and cognitive decline.

The second challenge in treating cognitive decline associated with these disorders is that the delivery of the drugs to the central nervous system (CNS) typically requires skilled medical or healthcare professionals. Many current treatment options are not safe and/or require IV administration by skilled physician in a clinic. Accordingly, improved compositions and methods for reducing and/or treating the cognitive impairment (learning, memory, or learning & memory) observed in APOE ε4 positive individuals showing AD, already experiencing onset of AD, or in vascular and/or other forms of dementia would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter provides a novel approach to protect cognitive function in individuals predisposed to AD by inhibiting the complement system at an early age. In some embodiments, the presently-disclosed subject matter is based, at least in part, on the discovery that intranasal administration of a humanized recombinant complement inhibitory protein (hrVCP) can improve cognitive function in a human APOE ε4 knock-in mouse model of AD. In one aspect, the presently-disclosed subject matter thus provides a method of protecting cognitive function in a subject predisposed to AD by administering to the subject a therapeutically effective amount of hrVCP. In some embodiments, the subject is a carrier of the APOE ε4 allele and the hrVCP can, in some embodiments, be administered intranasally. In another aspect, the presently-disclosed subject matter provides for early intervention with hrVCP such that its administration can have a positive impact on longevity in APOE ε4 carriers, and thereby extend the lifespan of APOE ε4 carriers, making their lifespan comparable to that of APOE ε2 and APOE ε3 carriers. In some embodiments, the achieved complement inhibition protects cognitive function independent of amyloid and tau pathology as targeting neuroinflammation has been found to be a therapeutic strategy in addressing AD pathogenesis.

The presently-disclosed subject thus provides, in some embodiments, a method of reducing an APOE ε4 mediated cognitive deficit that comprises administering to a subject in need thereof an effective amount of a hrVCP polypeptide. In some embodiments, the hrVCP polypeptide comprises a modified amino acid sequence including one or more amino acid substitutions to an amino acid sequence as set forth in SEQ ID NO: 2, wherein the one or more amino acid substitutions are selected from the group consisting of H98Y, E102K, E108K, E120K, and combinations thereof, provided at least one of the substitutions is E102K. In some embodiments, the hrVCP polypeptide exhibits a complement activation regulatory activity greater than a complement activation regulatory activity of a polypeptide comprising the amino acid sequence as set forth in SEQ ID NO: 2. In some embodiments, the hrVCP polypeptide has a modified amino acid sequence that comprises at least three of the amino acid substitutions selected from the group consisting of H98Y, E102K, E108K, E120K such as, in certain embodiments, the modified amino acid sequence comprising the amino acid sequence of SEQ ID NO: 3 or SEQ ID NO: 5. In certain embodiments, such hrVCP polypeptides are expressed in a mammalian cell, such as a Chinese Hamster Ovary (CHO) cell.

For administration of the hrVCP polypeptides, in some embodiments, administering the hrVCP polypeptide comprises intrathecal, spinal, subdural, or intravenous administration. In other embodiments, administering the hrVCP peptide comprises intranasal administration. In some embodiments, administering the hrVCP polypeptide intranasally comprises intranasal administration using an inhalation device, a nasal spray, or a nasal tube. In some embodiments, the hrVCP is administered with a pharmaceutically-acceptable vehicle, carrier, or excipient.

Further, with regard to the subjects administered the hrVCP polypeptides, in some embodiments, the subject is human. In some embodiments, the subject has dementia, such as Alzheimer's disease or vascular dementia. In this regard, in some embodiments, the presently-disclosed subject matter further provides methods of treating dementia characterized by an APOE ε4 mediated cognitive deficit. In some embodiments, such methods comprise intranasally administering to a subject in need thereof an effective amount of a hrVCP polypeptide comprising a modified amino acid sequence including one or more amino acid substitutions to an amino acid sequence as set forth in SEQ ID NO: 2, wherein the one or more amino acid substitutions are selected from the group consisting of H98Y, E102K, E108K, E120K, and combinations thereof, provided at least one of the substitutions is E102K. In some embodiments, the modified amino acid sequence comprises an amino acid sequence of SEQ ID NO: 3 or SEQ ID NO: 5.

Still further provided, in some embodiments, are methods of reducing complement activation mediated by APOE ε4 that comprise administering to a subject in need thereof an effective amount of a hrVCP polypeptide described herein. In some embodiments, the complement activation is alone or in combination with human TREM2. In some embodiments, the subject has a cognitive deficit secondary to aging, traumatic brain injury, or spinal cord injury.

Further features and advantages of the present invention will become evident to those of ordinary skill in the art after a study of the description, figures, and non-limiting examples in this document.

### BRIEF DESCRIPTION OF THE DRAWINGS

FIG. 1 shows the nucleic acid sequence (SEQ ID NO: 4) for the gene coding for the target hrVCP polypeptide as chemically synthesized with optimization for expression in mammalian cells.

FIGS. 2A-2B shows the amino acid sequence of a prior hrVCP protein (SEQ ID NO: 3, FIG. 2A) and an hrVCP protein of the presently-disclosed subject matter (SEQ ID NO: 5, FIG. 2B) including a signal sequence and produced from the nucleotide sequence of SEQ ID NO: 4.

FIGS. 3A-3D show the final hrVCP quality control (QC) samples using reduced PAGE with Coomassie blue staining. FIGS. 3A-3B are QC samples from medium and medium flow through (FT) respectively. FIG. 3C shows a Western Blot with Anti-Monkeypox virus/MPXV D14L, which is used to detect hrVCP. Polyclonal Antibody (ECL revelation). Ref #PTX18851-100. FIG. 3D shows final QC samples after endotoxin removal and lyophilization (2 μg per lane). MW designates a molecular weight marker.

FIG. 4 shows a final QC sample to detect hrVCP in medium using SDS-PAGE followed by the Coomassie blue staining (Each lane was 2 μg).

FIG. 5 is a graph showing body weight of all mice prior to treatment. Data represents mean±SEM. Not significant difference indicate P-value >0.05 by one-way ANOVA. Body weights of groups in the study prior to treatment are also shown in FIG. 5. One-way ANOVA found no significant differences in the body weights between the treatment groups prior to test.

FIG. 6 is a graph showing body weight of all mice at each week of treatment. Data represents mean±SEM. Not significant, by two-way ANOVA. Body weight was assessed weekly throughout the duration of the study as shown in FIG. 6. Two-way ANOVA found no significant treatment×time effect between groups such that the hrVCP formulation used was safe for intranasal administration and could be used in further studies.

FIGS. 7A-7B includes graphs showing a comparison of the mean escape latency during spatial learning paradigm. Data represents mean±SEM. *p<0.05, paired t-test between vehicle treated WT and human APOE4 knock-in groups for each day of the paradigm.

FIG. 8 is a graph showing time spent in each quadrant during the probe trial. Data represents mean±SEM. *p<0.05, **p<0.01, ***p<0.001, ****p<0.0001, Two-way ANOVA vs hrVCP treatment group (within treatment group).

FIG. 9 is a graph showing the number of virtual platform crossings during the probe trial. Data represents mean±SEM. *p<0.05, **p<0.01, ***p<0.001, ****p<0.0001, Two-way ANOVA vs hrVCP treatment group (within treatment group).

FIG. 10 is a graph showing the percent of virtual platform crossings in each quadrant during the probe trial. Data represents mean±SEM. *p<0.05, Paired t-test comparison between the APOE4-vehicle treated vs APOE4-hrVCP treatment group.

FIG. 11 is a graph showing the percent time spent near the walls during acquisition testing. Data represents mean±SEM. There was no significant difference using two-way repeated measures (RM) ANOVA.

FIG. 12 is a graph showing the average swim speed across acquisition trials. Data represents mean±SEM. Not significant difference with two-way RM ANOVA.

FIG. 13 shows representative sections of the H&E staining of the brain sections. The H&E staining of the brain sections includes sections from WT-mice and APOE4-vehicle mice.

FIG. 14 shows, by comparison, the silver staining of similar brain sections. The white matter of the cerebellum was stained using the Bielschowsky silver stain for the WT mouse (A at 20×), and APOE4-vehicle mouse (B at 20×). White matter is stained dark. The positive control for the tissue of the human brain showing amyloid plaques is shown in C. No plaques were observed in either WT or APOE4 groups.

FIG. 15 is a graphical representation of protection of the brain function by hrVCP and showing how complement regulation by intranasal hrVCP administration prevents neuroinflammation, subsequent neurodegeneration, and offers protection of cognitive function in neuroinflammatory disorders of the brain such as AD and vascular dementia. Briefly, the complement system is upregulated in APOE ε4 carriers suffering from AD or other forms of dementia including vascular dementia. This leads to neuroinflammation, neurodegeneration, and subsequent loss of cognitive function of the brain. hrVCP administered intranasally reduces this loss of cognitive function of the brain by virtue of its ability to regulate the complement system.

### BRIEF DESCRIPTION OF THE SEQUENCE LISTING

SEQ ID NO: 1 provides the nucleotide sequence encoding a VCP polypeptide isolated from a vaccinia virus (strain Western Reserve).

SEQ ID NO: 2 provides the amino acid sequence of the mature polypeptide (i.e., without the signal sequence) encoded by SEQ ID NO: 1.

SEQ ID NO: 3 is an amino acid sequence of an hrVCP polypeptide including H98Y, E102K, and E120K amino acid substitutions.

SEQ ID NO: 4 is a nucleotide sequence encoding a hrVCP polypeptide of the presently-disclosed subject matter and optimized for expression in mammalian cells.

SEQ ID NO: 5 is an amino acid sequence of an hrVCP polypeptide encoded by the nucleotide sequence of SEQ ID NO: 4 that was optimized for expression in mammalian cells and including H98Y, E102K, and E120K amino acid substitutions. Residues 1-20 of SEQ ID NO: 5 include a SIP IL2 signal sequence while residues 21-264 of SEQ ID NO: 5 include the hrVCP sequence of SEQ ID NO: 3.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

### Additional Definitions

The terms “associated with”, “operably linked”, and “operatively linked” refer to two nucleic acid sequences that are related physically or functionally. For example, a promoter or regulatory DNA sequence is said to be “associated with” a DNA sequence that encodes an RNA or a polypeptide if the two sequences are operatively linked, or situated such that the regulator DNA sequence will affect the expression level of the coding or structural DNA sequence.

The terms “coding sequence” and “open reading frame” (ORF) are used interchangeably and refer to a nucleic acid sequence that is transcribed into RNA such as mRNA, rRNA, tRNA, snRNA, sense RNA, or antisense RNA. In some embodiments, the RNA is then translated in vivo or in vitro to produce a polypeptide.

The term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction.

As is also known in the art, two sequences that hybridize to each other under a given set of conditions do not necessarily have to be 100% fully complementary. The terms “fully complementary” and “100% complementary” refer to sequences for which the complementary regions are 100% in Watson-Crick base-pairing, i.e., that no mismatches occur within the complementary regions. However, as is often the case with recombinant molecules (for example, cDNAs) that are cloned into cloning vectors, certain of these molecules can have non-complementary overhangs on either the 5′ or 3′ ends that result from the cloning event. In such a situation, it is understood that the region of 100% or full complementarity excludes any sequences that are added to the recombinant molecule (typically at the ends) solely as a result of, or to facilitate, the cloning event. Such sequences are, for example, polylinker sequences, linkers with restriction enzyme recognition sites, etc.

The term “expression cassette” refers to a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually encodes a polypeptide of interest but can also encode a functional RNA of interest, for example antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression.

Typically, however, the expression cassette is heterologous with respect to the host; i.e., the particular DNA sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus. In the case of a multicellular organism such as a plant, the promoter can also be specific to a particular tissue, organ, or stage of development.

The term “fragment” refers to a sequence that comprises a subset of another sequence. When used in the context of a nucleic acid or amino acid sequence, the terms “fragment” and “subsequence” are used interchangeably. A fragment of a nucleic acid sequence can be any number of nucleotides that is less than that found in another nucleic acid sequence, and thus includes, but is not limited to, the sequences of an exon or intron, a promoter, an enhancer, an origin of replication, a 5′ or 3′ untranslated region, a coding region, and a polypeptide binding domain. It is understood that a fragment or subsequence can also comprise less than the entirety of a nucleic acid sequence, for example, a portion of an exon or intron, promoter, enhancer, etc. Similarly, a fragment or subsequence of an amino acid sequence can be any number of residues that is less than that found in a naturally occurring polypeptide, and thus includes, but is not limited to, domains, features, repeats, etc. Also similarly, it is understood that a fragment or subsequence of an amino acid sequence need not comprise the entirety of the amino acid sequence of the domain, feature, repeat, etc.

A fragment can also be a “functional fragment”, in which the fragment retains a specific biological function of the nucleic acid sequence or amino acid sequence of interest. For example, a functional fragment of a transcription factor can include, but is not limited to, a DNA binding domain, a transactivating domain, or both. Similarly, a functional fragment of a receptor tyrosine kinase includes, but is not limited to a ligand binding domain, a kinase domain, an ATP binding domain, and combinations thereof.

The term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

The terms “heterologous”, “recombinant”, and “exogenous”, when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides.

A “homologous” nucleic acid (or amino acid) sequence is a nucleic acid (or amino acid) sequence naturally associated with a host cell into which it is introduced.

The term “inhibitor” refers to a chemical substance that inactivates or decreases the biological activity of a polypeptide such as a complement component.

The term “isolated”, when used in the context of an isolated DNA molecule or an isolated polypeptide, is a DNA molecule or polypeptide that, by the hand of man, exists apart from its native environment and is therefore not a product of nature. An isolated DNA molecule or polypeptide can exist in a purified form or can exist in a non-native environment such as, for example, in a transgenic host cell.

The term “mature polypeptide” refers to a polypeptide from which the transit peptide, signal peptide, and/or propeptide portions have been removed.

The terms “modified amino acid”, “modified amino acid sequence”, “modified polypeptide”, and “modified polypeptide sequence” refer to an amino acid sequence (or a polypeptide comprising that amino acid sequence) that is different from a native amino acid sequence (or a polypeptide that has such an amino acid sequence) that results from an intentional manipulation of the amino acid sequence or the nucleic acid sequence encoding the amino acid sequence. For example, an hrVCP is a modified polypeptide and comprises a modified amino acid sequence because it contains at least one amino acid substitution relative to a naturally occurring VCP amino acid sequence (see e.g., the naturally occurring sequence of VCP set forth in SEQ ID NO: 2 as compared to the modified amino acid sequences presented in SEQ ID NO: 3).

The term “native” refers to a gene that is naturally present in the genome of an untransformed cell. Similarly, when used in the context of a polypeptide, a “native polypeptide” is a polypeptide that is encoded by a native gene of an untransformed cell's genome.

The term “naturally occurring” refers to an object that is found in nature as distinct from being artificially produced by man. For example, a polypeptide or nucleotide sequence that is present in an organism (including a virus) in its natural state, which has not been intentionally modified or isolated by man in the laboratory, is naturally occurring. As such, a polypeptide or nucleotide sequence is considered “non-naturally occurring” if it is encoded by or present within a recombinant molecule, even if the amino acid or nucleic acid sequence is identical to an amino acid or nucleic acid sequence found in nature.

The term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Rossolini et al. (1994) MoI Cell Probes 8:91-98). The terms “nucleic acid” or “nucleic acid sequence” can also be used interchangeably with gene, open reading frame (ORF), cDNA, and mRNA encoded by a gene.

The terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long.

A fragment can retain one or more of the biological activities of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived.

The term “isolated”, when applied to a nucleic acid or polypeptide, also denotes that the nucleic acid or polypeptide is essentially free of other cellular components with which it is associated in the natural state. It can be in a homogeneous state although it can be in either a dry or aqueous solution. Homogeneity and whether a molecule is isolated can be determined using analytical chemistry techniques such as polyacrylamide gel electrophoresis or high performance liquid chromatography. A polypeptide that is the predominant species present in a preparation is substantially isolated. The term “isolated” denotes that a nucleic acid or polypeptide gives rise to essentially one band in an electrophoretic gel. Particularly, it means that the nucleic acid or polypeptide is in some embodiments at least about 50% pure, in some embodiments at least about 85% pure, and in some embodiments at least about 99% pure.

The terms “significant increase” or “greater than” refers to an increase in activity (for example, inhibition of complement activation or inhibition of the activity of a complement component) that is larger than the margin of error inherent in the measurement technique. In some embodiments an increase in inhibition by about 10% or greater over a baseline activity (for example, the inhibitory activity of a naturally occurring VCP versus the inhibitory activity of an hrVCP under a given set of conditions), in some embodiments an increase by about 20% or greater, in some embodiments an increase by about 25% or greater, and in some embodiments an increase by about 50% or greater is a significant increase in inhibitory activity.

The terms “significantly less” and “significantly reduced” refer to a result (for example, activation of complement or an activity of a complement component) that is reduced by more than the margin of error inherent in the measurement technique. In some embodiments a decrease in activation or activity by about 10% or greater over a baseline activity (for example, the activation of complement activity or the activity of a complement component in the presence of a naturally occurring VCP versus the same in the presence of an hrVCP under a given set of conditions), in some embodiments a decrease in activation or activity by about 20% or greater, in some embodiments a decrease in activation or activity by about 25% or greater, and in some embodiments a decrease in activation or activity by about 50% or greater is a significantly reduced activation of complement or activity of a complement component.

The term “subsequence” refers to a sequence of nucleic acids or amino acids that comprises a part of a longer sequence of nucleic acids or amino acids (e.g., polypeptide), respectively.

The term “transformation” refers to a process for introducing heterologous DNA into a cell. Transformed cells are understood to encompass not only the end product of a transformation process, but also transgenic progeny thereof.

The terms “transformed”, “transgenic”, and “recombinant” refer to a cell of a host organism such as a mammal into which a heterologous nucleic acid molecule has been introduced. The nucleic acid molecule can be stably integrated into the genome of the cell or the nucleic acid molecule can also be present as an extrachromosomal molecule. Such an extrachromosomal molecule can be auto-replicating. Transformed cells, tissues, or subjects are understood to encompass not only the end product of a transformation process, but also transgenic progeny thereof. A “non-transformed,” “non-transgenic”, or “non-recombinant” host refers to a wild type organism, e.g., a mammal or a cell therefrom, which does not contain the heterologous nucleic acid molecule.

For the purpose of some embodiments, ApoE ε4 or ApoE4 or APOE4 are mentioned. All these terms are used interchangeably to describe the allele ApoE ε4

### Hrvcp Polypeptides and Polynucleotides

The presently-disclosed subject matter provides a method of protecting cognitive function in subjects predisposed to disorders such as AD, particularly in those subjects carrying the APOE ε4 allele. The method comprises, in some embodiments, administering a therapeutically effective amount of a humanized recombinant complement inhibitory protein (hrVCP) to a subject.

Complement forms an important part of the innate immune system. It comprises about 30 proteins, some of which act within a cascade-like reaction sequence, while others serve as control proteins or as cellular receptors. For a review of the complement system, see Walport, M. J. (2001), N. Eng. J. Med., vol. 344, pp. 1058-1066 and Walport, M. J. (2001), N. Eng. J. Med., vol. 344, pp. 1140-1144, herein incorporated by reference. Certain components are present in the blood in precursor forms and must be activated. Complement can be activated by any of three pathways, (1) the antibody-dependent classical pathway (C1-C4-C2-C3), (2) the carbohydrate-dependent lectin pathway (MBL-C4-C2-C3), and (3) the alternative pathway (C3b-Factor B-C3), which is triggered directly by pathogen surfaces.

Activated complement has many functions, including initiation of inflammation, recruitment of leukocytes, clearance of immune complexes, neutralization of pathogens, regulation of antibody responses and cytolysis (the lytic pathway, via C5b-C6-C7-C8-C9, i.e., the membrane attack complex (MAC)). The complement system is a very powerful mediator of inflammation, and complement activation generates proinflammatory peptides such as the anaphylatoxins C3a and C5a, which recruit and activate leukocytes, the cell-bound opsonins C4b and C3b, which facilitate phagocytosis of the target, and MAC, i.e., C5b-9, which lyses target cells and may activate bystander cells to release pro-inflammatory mediators. Uncontrolled activation of complement and consequent host cell damage is prevented by a vast array of regulatory proteins, either circulating in plasma or expressed at the cell surface. However, in some circumstances, control of complement activation is inadequate or absent, which can result in deleterious effects, such as that seen in certain autoimmune and inflammatory diseases.

Vaccinia complement control protein (VCP) is a strong inhibitor of the classical, lectin and alternative pathways of complement, acting on both C4 and C3. VCP is a 35 kDa, soluble, secreted product of the vaccinia virus containing four short consensus repeats that share the greatest sequence homology with several proteins of the regulators of complement activity (RCA) family, including C4 binding protein (C4-bp; 38% identity), membrane cofactor protein (MCP; 35% identity) and decay-accelerating factor (DAF; 31% identity). SEQ ID NO: 1 provides the nucleotide sequence encoding a VCP polypeptide isolated from a vaccinia virus (strain Western Reserve). SEQ ID NO: 2 provides the amino acid sequence of the mature polypeptide (i.e., without the signal sequence) encoded by SEQ ID NO: 1. VCP shares the greatest functional similarity with complement receptor 1 (CR1). VCP binds to C4b, blocks the formation of the classical pathway C3 convertase, binds C3b, causes the accelerated decay of the classical pathway convertase, and blocks the conversion of C3 to C3b in both the classical and alternative pathways by promoting Factor I cleavage of C3b. Like its soluble mammalian RCA counterparts C4-bp and Factor H, but unlike the membrane RCA molecules decay accelerating factor (DAF), membrane cofactor protein (MCP), and soluble complement receptor 1 (CR1), it displays heparin-binding capabilities, suggesting an in vivo role in connection with heparan sulfate proteoglycans lining the endothelial cell layer. By blocking complement activation at multiple sites, VCP downregulates proinflammatory chemotactic factors (C3a, C4a, and C5a) resulting in reduced cellular influx and inflammation.

Further detailed description of VCP can be found in the following references, each of which is incorporated herein by reference: Kotwal, G. J. & Moss, B. (1988) Nature, vol. 335(6186), pp. 176-178; Kotwal, G. J. & Moss, B. (1989) J. Virol., vol. 63, pp. 690-696; U.S. Pat. Nos. 5,157,110; 5,187,268; Kotwal, G. J, et al. (1990) Science, vol. 250(4982), pp. 827-830; McKenzie, R. et al. (1992) J. Infect. Dis., vol. 166, pp. 1245-1250; Sahu, A. et al. (1998) J. Immunol., vol. 160, pp. 5596-5604; Smith, S A et al. (2000) J. Virol., vol. 74, pp. 5659-5666; Murthv, K. H. et al. (2001) Cell, vol. 104, pp. 301-311; and Smith, S A et al. (2003) Biochim. Biophys. Acta, vol. 1650, pp. 30-39. In view of the described functional characteristics of VCP, it is considered to be a well-established molecule for inhibiting complement activation and reducing complement-mediated inflammation.

In some embodiments, the presently-disclosed subject matter makes use of an isolated recombinant vaccinia virus complement control protein (hrVCP) polypeptide. The hrVCP polypeptide comprises in some embodiments a modified amino acid sequence comprising one or more amino acid substitutions to an amino acid sequence of a naturally occurring VCP polypeptide, such as for example the polypeptide sequence as set forth in SEQ ID NO: 2, wherein the hrVCP polypeptide exhibits a complement activation regulatory activity greater than a complement activation regulatory activity of a naturally occurring VCP polypeptide. Modifications to the naturally occurring VCP polypeptides can be carried out using techniques known in the art, including site-specific mutagenesis. Site-specific mutagenesis (also referred to as “site-directed mutagenesis”) is a technique useful in the preparation of individual peptides, or biologically functional equivalent proteins or peptides, through specific mutagenesis of the underlying DNA. The technique further provides a ready ability to prepare and test sequence variants; for example, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 30 nucleotides in length is employed, with about 5 to 10 residues on both sides of the junction of the sequence being altered.

In general, the technique of site-specific mutagenesis is well known in the art (see e.g., Adelman et al. (1983) DNA 2:183; Sambrook & Russell (2001) Molecular Cloning: A Laboratory Manual, Third Edition, Cold Spring Harbor Laboratory Press, Cold Spring Harbor, New York) and can be achieved in a variety of ways generally known to those of skill in the art. In some embodiments, the one or more amino acid substitutions are selected from the group consisting of H98Y, E102K, E108K, E120K, and combinations thereof. Further, in some embodiments, the modified amino acid sequence comprises an amino acid sequence of SEQ ID NO: 3 or SEQ ID NO: 5. Additional modified hrVCP sequences capable of use in the present invention are described, for example, in U.S. Pat. No. 7,749,963, which is incorporated by reference herein in its entirety.

As discussed above, VCP exhibits complement activation regulatory activity. For example, VCP binds to C4b, blocks the formation of the classical pathway C3 convertase, binds C3b, causes the accelerated decay of the classical pathway convertase, and blocks the conversion of C3 to C3b in both the classical and alternative pathways by promoting Factor I cleavage of C3b. As such, by blocking complement activation at multiple sites, VCP downregulates proinflammatory chemotactic factors (C3a, C4a, and C5a) resulting in reduced cellular influx and inflammation. Through selected modifications of the amino acid sequence of a VCP, such as, for example, a VCP having the amino acid sequence set forth in SEQ ID NO:2, the presently disclosed subject matter provides and makes use of modified recombinant polypeptides (hrVCP) comprising one or more amino acid substitutions (i.e., changing an amino acid at a given position in SEQ ID NO: 2 to a different amino acid) exhibiting enhanced complement activation regulatory activity, as compared to complement activation regulatory activity found in the VCP from which the hrVCP was derived. In some embodiments therefore, the complement activation regulatory activity comprises regulating activation of a classical complement activation pathway, an alternative complement activation pathway, or both the classical and alternative complement activation pathways. Further, in some embodiments, regulating activation of the classical complement activation pathway, the alternative complement activation pathway, or both the classical and alternative complement activation pathways comprises inhibiting activation of at least one complement component, inhibiting activity of at least one activated complement component, or combinations thereof. Further, in some embodiments, the complement component comprises C3 or C4 and the activated complement component comprises C3b or C4b.

Measuring a change in complement activation regulatory activity can be achieved by any of several techniques related to measuring complement activation and activity as are generally well known in the art. For example, activation of the classical complement pathway can be measured using a sensitized sheep red blood cell assay and activation of the alternative complement pathway can be measured using an immunoassay that measures complement factor Bb formation.

The presently disclosed subject matter further provides isolated nucleic acids encoding an hrVCP polypeptide as disclosed herein above.

Method of Producing hrVCP

The presently-disclosed subject matter also provides methods of producing an hrVCP polypeptide having enhanced complement activation regulatory activity. In some embodiments, the method comprises providing a naturally occurring VCP polypeptide, such as for example a naturally occurring VCP polypeptide comprising an amino acid sequence as set forth in SEQ ID NO: 2, and substituting one or more amino acids of the VCP polypeptide amino acid sequence to produce an hrVCP polypeptide having enhanced complement activation regulatory activity when compared to a complement activation regulatory activity of the naturally occurring VCP polypeptide. In some embodiments, site-specific mutagenesis is utilized, as discussed in detail herein above, to substitute one or more amino acids of the naturally occurring VCP to produce a novel hrVCP. In some embodiments, the modified amino acid sequence is produced by making use of a nucleic acid sequence or gene encoding a desired hrVCP modified amino acid sequence and that is introduced into an expression cassette for expression in a desired cell, such as Chinese Hamster Ovary (CHO) cells or any other mammalian or microbial cells such as HEK293, Pichia pastoris, yeast, or E. coli cells. As such, in some embodiments, the presently-disclosed subject matter further provides cells, such as the foregoing, including such constructs.

### Therapeutic Methods and Pharmaceutical Formulations

Alzheimer's disease (AD) is still one of the most complex disorders of the brain, even 118 years after the term AD was coined to acknowledge the efforts of Dr. Alois Alzheimer who had documented amyloid plaque and neurofibrillary tangles (NFT), as pathological hallmarks of AD. It took more than 100 years of scientific efforts to get the first-generation U.S. FDA approved monoclonal antibodies to clear amyloid plaques. Although, the U.S. FDA has recently approved anti-amyloid therapies, aducanumab application was withdrawn in Europe in 2022, and in July 2024, the European Medicinal Agency (EMA) rejected approval to lecanemab (a U.S. FDA approved drug) citing that risk due to adverse events, particularly, microhemorrhage outweighs delaying cognitive decline in AD. The much-awaited panacea for AD has shown a serious side effect of microhemorrhage in a significant number of AD patients, particularly in homozygous and/or heterozygous carriers of APOE ε4 allele. Considering that there are no safe and effective treatment against the primary hallmarks in AD, neuroinflammation is now the focus of research and has indeed become the third hallmark of AD and is target for future drug development in addition to tauopathies. Gut microbiome and lifestyle intervention also play a role in AD. However, these can be supportive therapy and not the primary target to develop future drug therapies. In this regard, and without wishing to be bound by any particular theory or mechanism, the complement system is believed to play an important role in mediating neuroinflammation and numerous roles for complement in AD and related disorders are appreciated, including: neuroprotection in C3 deficient plaque-rich APP/PS1 mice, an upregulation in the complement system to mediate neuroinflammation in AD, that key pathological hallmarks of AD such as amyloid plaques, oligomers, phosphorylated tau and NFT activate the complement system in AD, and that Apolipoprotein E (ApoE) levels are even more than that of Tau, APP/Aβ, and a-Synuclein peptides increased in the brains of AD patients. In particular, it is appreciated that ApoE peptides are strongly enriched in dementia cases, including from individuals lacking the APOE ε4 genotype. Moreover, the APOE protein level has been observed to be robustly associated with levels of complement proteins C3 and C4, indicating indicates a link between APOE4 and complement components in cognitive deficit associated with AD pathogenesis. Briefly, APOE ε4 and complement system are believed to work in synergy in AD to mediate the neuroinflammation along with amyloid and Triggering receptor expressed on myeloid cells 2 (TREM2), and the role of complement in mediating neuroinflammation via direct and indirect upregulation of complement components.

Accordingly, and again without wishing to be bound by any particular theory or mechanism, it is believed that considering the role of complement system in AD, the use and development of a safe and effective complement regulatory therapy is a suitable option available to meet this unmet need to control neuroinflammation in AD. It is believed that in some embodiments, such a therapeutic strategy is particularly helpful for the APOE ε4 positive individuals who are more prone to the serious adverse events in response to anti-amyloid antibodies. To this end, it has now been surprisingly discovered that VCP is effective in offering protection to an aging brain including after being administered intranasally, and it has also been discovered that early administration prevents cognitive decline at a later age in transgenic mouse models of amyloid pathology. To further increase efficacy of VCP, three surface amino acids were changed from its structure. A humanized recombinant VCP (hrVCP; e.g., SEQ ID NO: 3) was found to be several fold more potent in inhibiting complement system as compared to VCP in hemolysis assay performed using normal human serum (NHS) as source of complement. For the current investigation described herein below, hrVCP was tested using the human APOE ε4 knock in mice (LOAD2) and it was discovered that hrVCP was beneficial in reducing cognitive deficits observed in those mice and mediated by APOE ε4.

The presently-disclosed subject thus provides, in some embodiments, a method of reducing an APOE ε4 mediated cognitive deficit that comprises administering to a subject in need thereof an effective amount of a hrVCP polypeptide.

An “APOE ε4 mediated cognitive deficit” and grammatical variations thereof refers to cognitive deficits, such a deficits in learning, memory, or combinations thereof, observed in a subject due to the presence of the APOE ε4 allele in the subject relative to those subject that do not include the APOE ε4 allele. It is appreciated that APOE ε4 mediated cognitive deficit can occur at a faster rate than that of non APOE ε4 subjects and can manifest as an early onset dementia, and can affect multiple domains/functions, and manifest as episodic memory (e.g. difficulties with learning and recalling new information, events, experiences etc.), episodic functions (challenges with planning, problem-solving, cognitive flexibility etc.) or spatial navigation/orientation. Such a deficit after confirming the presence of APOE ε4 allele through genetic testing is often performed using the cognitive tests such as, but not limited to: 1) Mini-Mental State Examination (MMSE), a widely used screening tool for assessment of cognitive function with respect to orientation, memory, attention, language, and visuospatial skills; 2) Montreal Cognitive Assessment (MoCA), which is considered to be more sensitive than that of MMSE, especially for mild cognitive impairment (MCI), and which evaluates a broader range of cognitive domains, including executive function, visuospatial abilities, and memory; 3) Alzheimer's Disease Assessment Scale-Cognitive Subscale (ADAS-Cog), which is specifically designed for AD, and measures memory, language, praxis, attention, and other cognitive abilities. Of course, the foregoing list of cognitive test is non-exhaustive as other tests can also be utilized by those skilled in the art to determine cognitive function, including, but not limited to: neurophysical assessments, functional assessments and/or imaging techniques, such as magnetic resonance imaging (MRI) or positron emission tomography (PET) to measure cognitive deficit. Physicians and other skilled in the art can also utilize any questionnaire based tests and/or tools to define the scale of the cognitive deficit. In some embodiment, APOE ε4 mediated inflammation can be a primary cause of the cognitive deficit or associated disorder, such as AD, vascular dementia, or similar disorder, or the APOE ε4 mediated inflammation can be a mediating factor in the persistence of the disorder.

In some embodiments of the therapeutic methods, the hrVCP polypeptide utilized comprises a modified amino acid sequence including one or more amino acid substitutions to the amino acid sequence as set forth in SEQ ID NO: 2, wherein the one or more amino acid substitutions are selected from the group consisting of H98Y, E102K, E108K, E120K, and combinations thereof, provided at least one of the substitutions is E102K. In some embodiments, the hrVCP polypeptide exhibits a complement activation regulatory activity greater than a complement activation regulatory activity of a polypeptide comprising the amino acid sequence as set forth in SEQ ID NO: 2. In some embodiments, the hrVCP polypeptide has a modified amino acid sequence that comprises at least three of the amino acid substitutions selected from the group consisting of H98Y, E102K, E108K, E120K such as, in certain embodiments, the modified amino acid sequence comprising the amino acid sequence of SEQ ID NO: 3 or SEQ ID NO: 5. In certain embodiments, such hrVCP polypeptides are expressed in a mammalian cell such as a Chinese Hamster Ovary (CHO) cell.

In some embodiments, the subject has dementia, such as Alzheimer's disease or vascular dementia. In this regard, in some embodiments, the presently-disclosed subject matter further provides methods of treating dementia characterized by an APOE ε4 mediated cognitive deficit. In some embodiments, such methods comprise intranasally administering to a subject in need thereof an effective amount of a recombinant vaccinia virus complement control protein (hrVCP) polypeptide comprising a modified amino acid sequence including one or more amino acid substitutions to an amino acid sequence as set forth in SEQ ID NO: 2, wherein the one or more amino acid substitutions are selected from the group consisting of H98Y, E102K, E108K, E120K, and combinations thereof, provided at least one of the substitutions is E102K. In some embodiments, the modified amino acid sequence comprises an amino acid sequence of SEQ ID NO: 3 or SEQ ID NO: 5.

Still further, in some embodiments, are methods of reducing complement activation mediated by APOE ε4 that comprise administering to a subject in need thereof an effective amount of a recombinant vaccinia virus complement control protein (hrVCP) described herein. In some embodiments, the complement activation is alone or in combination with human TREM2. In some embodiments, the subject has a cognitive deficit secondary to aging, traumatic brain injury, or spinal cord injury.

The terms “treatment” or “treating,” as used herein, refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; treatment directed to minimizing or partially or completely inhibiting the development of the associated disease, pathological condition, or disorder; and supportive treatment, that is, treatment employed to supplement another therapy directed toward the improvement of the associated disease, pathological condition, or disorder.

The terms “reducing,” “reduction,” “inhibiting,” “inhibition” and grammatical variations thereof do not necessarily refer to the ability to completely inactivate all target biological activity in all cases. Rather, the skilled artisan will understand that those terms refer to decreasing biological activity of a target, such as can occur when a ligand binds a site of the target, a protein in a biochemical pathway of the target is blocked, a non-native complexes with a target, or the like. Such decrease in biological activity can be determined relative to a control, wherein the control can be representative of an environment in which an inhibitor is not administered. For example, in some embodiments, a decrease in activity relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. In some embodiments, the increases and/or decreases described herein can be in reference to a control subject having cognitive deficits and that has not been treated with one of the presently-disclosed polypeptides. In other embodiments, the increases and/or decreases described herein can be in reference to a baseline obtained in a subject that is in need of treatment, but has not yet began a particular therapeutic regimen.

For administration of a therapeutic composition as disclosed herein (e.g., a hrVCP polypeptide of SEQ ID NO: 3), conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments, administering the hrVCP polypeptide comprises intrathecal, spinal, subdural, or intravenous administration. In other embodiments, administering the hrVCP peptide comprises intranasal administration. In some embodiments, administering the hrVCP polypeptide intranasally comprises intranasal administration using an inhalation device, a nasal spray, or a nasal tube, or any other device commonly used to administer drugs intranasally.

In some embodiments, as indicated above, the intranasal administration can be replaced with any other technique of administration where drug reaches the cerebrospinal fluid (CSF) or into the central nervous system (CNS). Some of the known “parenteral” routes of such administration include but are not limited to intravenous, intracerebroventricular, intrathecal, subdural intramuscular, intra-arterial injection, infusion and any other techniques for administration into CNS or CSF.

Regardless of the route of administration, the compositions of the presently-disclosed subject matter are typically administered in amount effective to achieve the desired response. As such, the term “effective amount” is used herein to refer to an amount of the therapeutic composition (e.g., a hrVCP polypeptide of SEQ ID NO: 3 and a pharmaceutically vehicle, carrier, or excipient) sufficient to produce a measurable biological response (e.g., a decrease in APOE ε4 mediated cognitive decline or inflammation). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art. One skilled in the art can readily assess the potency and efficacy of a candidate compound of the presently disclosed subject matter and adjust the therapeutic regimen accordingly. In some embodiments, for example, a therapeutically effective amount of a complement inhibitor for the treatment and/or prophylaxis of APOE ε4 mediated cognitive decline or inflammation is from about 0.01 g/kg to about 0.1 g/kg per dose. In some embodiments, and as described in detail below, the hrVCP is formulated for nasal administration in sterile, isotonic, pH balanced aqueous buffered solution and can be administered at least over a period of month (250 μL once daily for up to 10 to 20 min) with the concentration ranging from 1 μg/μl using a polypropylene tubing or other intranasal device such as an intranasal metered syringe.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Florida; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pennsylvania; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

In some embodiments, the hrVCP is administered with a pharmaceutically-acceptable vehicle, carrier, or excipient. In some embodiments, the compound is administered parenterally or intranasally as a pharmaceutical formulation in dosage unit formulations containing standard, well-known nontoxic pharmaceutically acceptable carriers, excipients, adjuvants, and vehicles as desired.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

Preferred carriers include neutral saline solutions buffered with phosphate, lactate, Tris, and the like. The excipients used in the current embodiment to prepare the formulation are known to be inactive and the preservatives, stabilizers, surfactants can be used to protect the biological activity of hrVCP. The nasal formulations such as mucolytics and adhesives are inert excipients that when added will not alter the biological activity of hrVCP.

Of course, one of skill would know to purify the carrier and therapeutic compound sufficiently to render it essentially free of undesirable contaminants, such as endotoxins and other pyrogens such that it does not cause any untoward reactions in the subject receiving the formulation. In the some embodiments, the hrVCP is formulated in a sterile, aqueous, isotonic, pH buffered formulation containing, boric acid, sodium borate, potassium chloride, sodium chloride preserved with polyaminopropide biguanide and edetate disodium.

Further, with regard to the subjects administered the hrVCP polypeptides, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like. In some embodiments, the subject is human.

In some embodiments, the presently-disclosed subject matter provides a method of treating individuals predisposed to AD, vascular dementia and other forms of dementia, particularly those carrying the APOE ε4 allele as APOE ε4 is believed to be a strong genetic risk factor for AD and as carriers of the APOE ε4 allele are at a significantly increased risk of developing AD and experiencing more severe disease progression. In some embodiments, early intervention with hrVCP in APOE ε4 carriers protects cognitive function and potentially delays the onset of AD. In some embodiments, intranasal administration of hrVCP improves cognitive function even in the absence of amyloid and tau pathology.

In some embodiments, the presently-disclosed subject matter provides that early intervention with hrVCP has a positive impact on longevity in APOE ε4 carriers, such that early intervention can potentially extend the lifespan of APOE ε4 carriers, making it comparable to that of APOE ε2 and APOE ε3 carriers.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples.

### EXAMPLES

### Materials & Methods

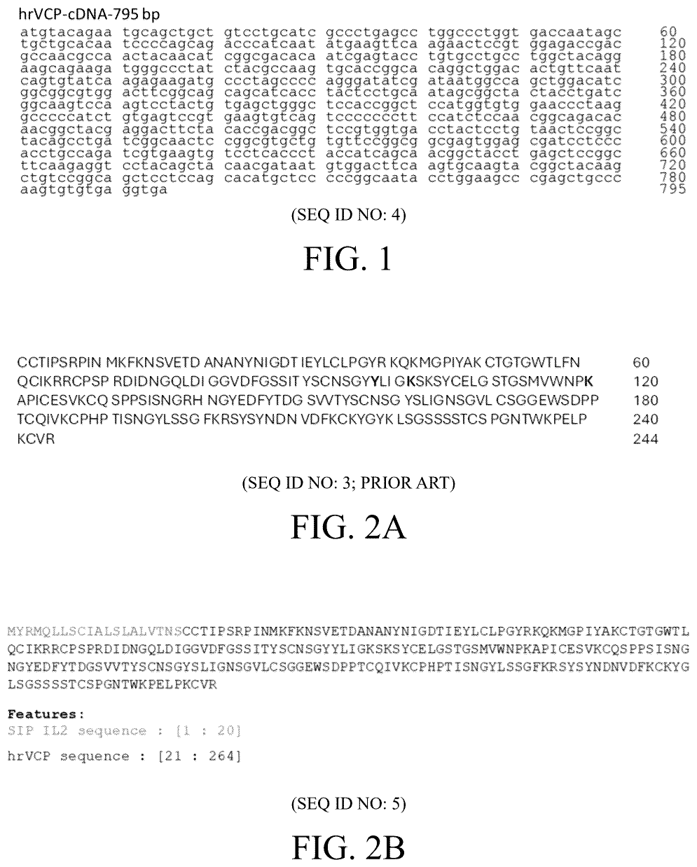

Genes coding for the target proteins: The gene coding for the hrVCP was chemically synthesized with optimization for expression in mammalian cells, including Chinese Hamster Ovary (CHO) cells, using the sequence as shown in FIG. 1.

Expression vectors: The cDNA sequence described above (hrVCP-cDNA-795 pb) was subcloned into an established mammalian cells expression vector pTXs1 (ProteoGenix, Newark, DE). The expected protein was produced, and included the changed model structure describe above. The expression was performed employing the established expression vector pTXs1 construction with transfection into XtenCHOTM (Chinese Hamster Ovary) cells (100 ml culture) using an established Xten transient transfection protocol (ProteoGenix). Culture medium and cells were collected 7 days post-transfection, and were further analyzed using SDS-PAGE and were purified. For purification, small scale (˜160 ml) cell culture medium was collected, and purification was performed by affinity purification using a heparin purification column using a standard protocol. Binding and washing were performed using Tris-buffered saline (TBS; pH 8). Elution was performed using sodium chloride (NaCl) concentration shift. After purification, hrVCP was analyzed by SDS-PAGE. Final sample QC testing was performed using qualitative analysis by SDS-PAGE and quantitative estimation of the protein concentration was performed by the Bradford method. For the purification from the medium, the wash fraction was pooled and buffer exchange with PBS, pH 7.5 was performed by dialysis method and with filtration. For purification from medium flow through, elution fractions were selected and the select elution fractions were pooled, and buffer exchange by dialysis method and filtration was again used. Subsequently, hrVCP was purified and endotoxin was removed and hrVCP solution was lyophilized before further processing and transport. After this endotoxin level was measured, purity and identity was confirmed using SDS-PAGE.

Test Article: Test article was hrVCP aqueous solution (1 μg/μL) and was administered intranasally once a day at a volume of 10 μL/nostril (total volume 20 μL per mouse).

Vehicle solution for intranasal administration: The test compound was diluted in the aqueous isotonic, pH balanced, sterile solution with the composition as described in the formulation section. This formulation without hrVCP was described as vehicle or saline without the test compound and was administered to the wild type (non-transgenic) and transgenic mice (APOE4) for the current experiment. Thus, the terms “vehicle” “saline” and aqueous formulation were used interchangeably for the purpose of these experiments. Mice were treated from 5 to 6 months of age at the time dosing for the Morris Water Maze (MWM) experiments described below.

In alignment of this protocol and also to test the safety of this solution (referred to as vehicle throughout the study), the vehicle was administered intranasally once a day at a volume of 10 μL per nostril. Mice from both the wild type and APOE ε4 knock-in groups were treated in a similar manner for approximately a month from 5-6 months of age. In addition to comparison between the groups, this aqueous solution without any active ingredient was also tested for its safety in animals.

Formulation of hrVCP for Intranasal Administration: For the current experiment, lyophilized hrVCP was suspended in aqueous, sterile, isotonic formulation containing preservatives (“vehicle”). However, it can be formulated in the aqueous solution, emulsion and suspension containing excipients with no known biological action and either additional excipients, such as mucolytics, can be used to increase the bioavailability and/or a different preservative or buffer system can be used. hrVCP can also be formulated using any known formulations in emulsions, nano-emulsions, suspension, cream etc. After that, it could be administered intranasally using a polyethylene tube or similar material, intranasal spray or inhalational spray with or without use of known special devices such as spacer. The concentration of hrVCP utilized was 1 μg/μL. Approximately 1 μg/1 g of body weight of mice was administered and found to be safe and effective at this concentration.

Animal Studies: The study in mice was performed at an Assessment and Accreditation of Laboratory Animal Care (AAALAC) accredited facility. Procedures were approved by the Institutional Animal Care and Use Committee in accordance with the National Institutes of Health (NIH) Guide for the Care and Use of Laboratory Animals. The integrity of the data was ensured through a quality control process.

Details of the transgenic strain: Female B6.Cg-Apoetm1.1(APOE*4)Adiuj Appem1Adiuj Trem2em1Adiuj/J (n=15 in each group; Jackson Laboratory; referred to hereinafter as APOE4 or APOE4 human knock-in mice), JAX stock #030670 RRID:IMSR_JAX:030670 Common Name: hAbeta/APOE4/Trem2*R47H, LOAD2 hAbeta/APOE4/Trem2*R47H is a triple mutant strain carrying a humanized ApoE knock-in mutation (sequence coding for isoform E4), a CRISPR/Cas9-generated App allele with a humanized Abeta1-42 region (G601R, F606Y, R609H in the mouse gene, corresponding to amino acid positions 676, 681, 684 in the human APP locus) and a CRISPR/Cas9-generated R47H point mutation of the Trem2 gene. These mice are thus suitable for use in studies related to Alzheimer's disease, lipoproteins, arteriosclerosis, and coronary heart disease (see Kotredes, K. P. et. al. (2024), which is incorporated herein by reference for full characterization data on these mice). The wild type (WT, non-transgenic) C57BL/6J mice were also supplied by the Jackson Laboratory.

Testing of intranasal administration of hrVCP in with APOE ε4 knock-in mouse model of Alzheimer's disease (AD): It is believed that hrVCP has never been tested in an animal model of AD, dementia or other forms of vascular dementia with APOE ε4 variant. To this end, as indicated above, the transgenic mice Strain B6.Cg-Apoetm1.1(APOE*4) Adiuj Appem #1Adiuj Trem2em1Adiuj/J was procured from Jackson Laboratory. This represented the best available mouse model of AD reflecting APOE ε4 and other pathophysiological features similar to AD. Again, this triple mutant strain carried a humanized ApoE knock-in mutation (sequence coding for isoform E4), a CRISPR/cas9-generated 94 bp deletion in exon 14 of the APP gene and a CRISPR/cas9-generated R47H point mutation of the Trem2 gene. The targeted Trem2 gene encodes a protein that is part of a receptor signaling complex with TYRO protein tyrosine kinase binding protein, and that activates macrophages and dendritic cells during immune responses. The TREM2 R47H mutation is a missense mutation in exon 2 that is one of the strongest genetic risk factors for late-onset Alzheimer's disease. Thus, in some of the embodiments, it was believed that the hrVCP could be used to control APOE ε4 mediated neurodegeneration and subsequent cognitive dysfunction.

The mouse model used in the current embodiment was also believed to be useful to evaluate APOE ε4 mediated cognitive deficits in AD, vascular dementia, cardiovascular diseases, and/or lipoprotein metabolism as described on the website of Jackson Laboratories for this strain and where it is indicated that the “ . . . triple mutant strain carries a humanized ApoE knock-in allele, in which exons 2, 3 and most of exon 4 of the mouse Apoe gene were replaced by human APOE4 gene sequence including exons 2, 3 and 4 (and some 3′ UTR sequence); a mutant allele of the App gene containing a 94 bp deletion in exon 14; and a knock-in of a point mutation into mouse Trem2, triggering receptor expressed on myeloid cells 2, gene containing a R471H point mutation, with two silent mutations. The targeted Apoe gene encodes apolipoprotein E, which is important in lipoprotein metabolism and cardiovascular disease as well as Alzheimer's disease, immunoregulation and cognition. The targeted App gene encodes amyloid beta precursor protein, a transmembrane cell surface receptor that is cleaved by secretase. Mutations in this gene have been associated with Alzheimer's disease. The targeted Trem2 gene encodes a protein that is part of a receptor signaling complex with TYRO protein tyrosine kinase binding protein, and that activates macrophages and dendritic cells during immune responses. The TREM2 R47H mutation is a missense mutation in exon 2 that is one of the strongest genetic risk factors for late-onset Alzheimer's disease. Mice that are homozygous for the Apoe^{tm1.1t(APOE*4)Adiuj} and Trem2^{em1 Adiuj} alleles, and heterozygous for the App^{em2 Adiuj} allele are viable and fertile.”

As described below, and by administering hrVCP, an improvement in APOE ε4 mediated cognitive decline was observed in the aforementioned mouse model. It was also observed that this mouse model develops cognitive dysfunction independent of amyloid plaques and tauopathy.

Animal housing: Female B6.Cg-Apoetm1.1(APOE*4)Adiuj Appem1Adiuj Trem2em1Adiu/J) mice and WT mice were supplied at 10 weeks of age. Upon receipt, mice were assigned unique identification numbers (tail marked) and group housed in Opti-MICE ventilated cages (Animal Care Systems, CO). Mice were housed in the same-genotype groups. Any mice that showed aggressive behavior were single housed. All animals were examined and weighed prior to initiation of the study to assure adequate health and suitability. Mice were balanced by body weight for each cage and cages were randomly assigned to the experimental treatments. During the study, 12/12 light/dark cycles were maintained. The room temperature was maintained between 20° C. and 23° C. with a relative humidity maintained around 50%. Chow and water were provided ad libitum for the duration of the study unless noted. Mice in each cage received the same treatment.

### Treatment Groups:

WT-Vehicle: Wild type (non-transgenic) C57BL/6J mice; Sex: female; n=14. There were 15 mice in this group, one was excluded due to fight injury.

APOE4-Vehicle: APOE4 knock-in mice that is B6. Cg-Apoetm1.1(APOE*4) Adiuj Appem #1Adiuj Trem2em1Adiuj/J; n=14 female mice; n=14 APOE4-treatment: Refers to a group of mice to which hrVCP was administered intranasally (10 ml each nostril for one month using 10 ml pipette without any anesthesia instead of vehicle in the control groups); 1 mg/mL reconstituted in the vehicle; n=15).

APOE4-treatment and APOE4-hrVCP: refers to the same group.

Morris Water Maze (MWM) Protocol Methods and Testing: Upon commencement of dosing, body weight was assessed weekly for the duration of the study. General health was assessed daily. Mice were balanced by body weight among the treatment groups. The MWM test assesses long term memory for place learning using visual cues and is dependent on hippocampal function and cerebellum as mice have to navigate and identify the platform based on the spatial cues in the testing room. Mice are trained to remember particular cues and their spatial arrangement predicts the position of an escape platform. This training is known as spatial learning. This is followed by a probe trial which is performed after the 5 days of training without inclusion of the escape platform. MWM is a standard and well-established protocol for testing learning and memory in rodents. It has been employed for evaluating many mice models of AD as well as for development of AD treatment. Thus, it was used for the current study. The test is performed in a circular tank, measuring 48″ in diameter. The water was filled to 24-25″ of the tank and was made opaque using white non-toxic aqueous paint. The water temperature was maintained at 25° C.±1° C. Extra-maze cues were mounted around the water tank. Trials were video recorded and analyzed by a computer program (Water Maze; Actimetrics, Wilmette, IL).

Protocol: On 5 consecutive days, test sessions were conducted with the platform submerged approximately 1.3 cm below the surface of opaque water. On each day, there were 4 trials each of 60 s duration and with an approximately 15 min gap between each trial. On the fifth day of acquisition training, the last trial (out of 4) consisted of a 60 s probe trial without the platform. The platform position and release points were counterbalanced across treatment groups such that half of the animals have the platform on the right side of the tank while the other half have it on the left side of the tank (this is done so there is no bias between the placement of the cues). The measurements included: time to reach the platform (training trials only); distance traveled to reach the platform (training trials only); percent time spent in the four quadrants of the maze (probe trials); percent and total number of crossings in the four quadrants of the maze (probe trials); thigmotaxis (training and probe trials); and swim speed (training and probe trials). Each of the mice were tested for their cognitive abilities using MWM approximately at the age of six months.

Statistical Analysis: Data were analyzed by analysis of variance (ANOVA) followed by Dunnett's post-hoc comparisons, where appropriate. Otherwise, t-test was performed for pair-wise comparison. An effect was considered significant if p<0.05. Data were represented as mean and standard error to the mean (s.e.m). One mouse from the WT group was excluded from treatment due to fight wounds. Only one data point (data 4 mean escape latency for hrVCP treatment group) was masked as it was an outlier. The statistical analyses were performed and independently evaluated using the trial version of JMP statistical software (Cary, NC).

Biological Sample Collections for histological studies: After completion of testing, brain samples were collected from both WT-vehicle and APOE4-vehicle treated mice. The brain tissue was collected and further processed for histological studies. For this, mice were deeply anesthetized with isoflurane. The abdominal cavity was opened, and a blunt end perfusion needle was inserted into the left ventricle and forwarded toward the ascending aorta. Immediately after, a small incision was made on the right atria. The perfusion needle was connected to an automated perfusion pump through a catheter system for a continuous administration of phosphate-buffered saline (PBS; 20 mM, pH 7.4 at room temperature). Whole brains were collected and split into 2 hemispheres. The right hemisphere was immersion fixed directly after perfusion and dissection overnight at 4° C. in freshly prepared 1×PBS containing 4% paraformaldehyde (PFA) in 15 ml tubes. After post-fixation, tissue was transferred to PBS and stored at 4° C. Out of the 14 female mice were used for the study, the brain tissue samples of 8 to 9 mice from WT-Vehicle and APP/PS1-Vehicle groups were collected and further processed for the histological staining. Following paraffin embedding of the brain tissue, the tissue sections were stained using hematoxylin and eosin (H& E staining) and silver staining (Bielschowsky's silver stain). Brain sections from human patients (collected after death) were supplied by a collaborator. The histology and immunohistochemistry samples were performed as per the standard procedure at the Jewish Hospital Pathology Laboratory, University of Louisville-Health, KY, USA. For H&E, the Leica histoscore spectra was used. For silver, the Polyscientific R&D corporation, Kit item #k080 was used. The staining was performed manually or using the Leica biosystem. The different sections after silver and H&E sections were observed using Olympus BX40 microscope and photographed using EPview software.

Short protocol description for purification: Small scale (˜160 ml) cell culture medium was collected, and purification was performed by affinity purification using a heparin purification column using a standard protocol. Binding and washes were performed using Tris-buffered saline (TBS; pH 8). Elution was performed using sodium chloride (NaCl) concentration shift. After purification, hrVCP was analyzed using SDS-PAGE.

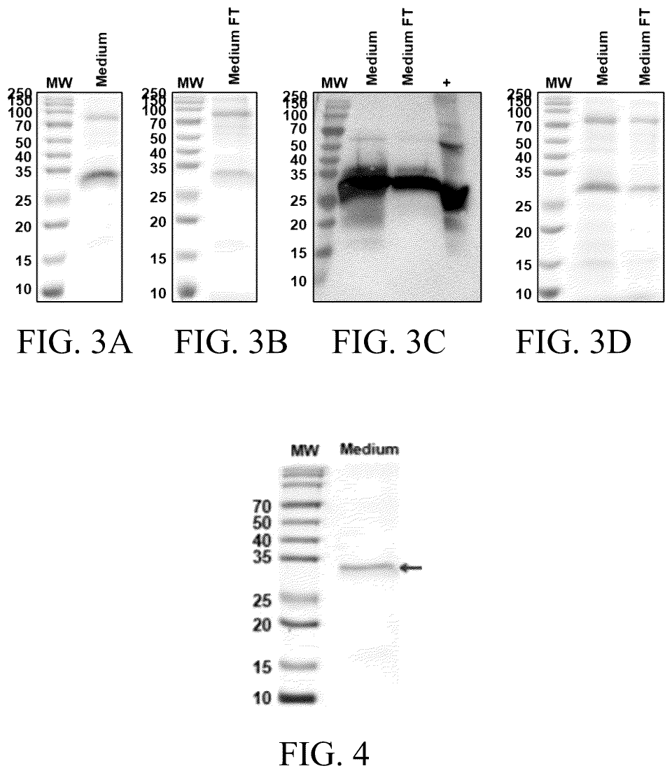

Final sample QC was performed using qualitative analysis by SDS-PAGE and quantitative estimation of the protein concentration by the Bradford method. For the purification from medium, wash fraction was pooled and buffer exchange with PBS, pH 7.5 was performed by the dialysis method and filtered. For the purification from medium flow through, elution fractions were selected and the select elution fractions were pooled, and buffer exchange by dialysis method was used with filtering. Subsequently, hrVCP was purified and endotoxin was removed and hrVCP solution was lyophilized before further processing/transport. After this endotoxin level was measured, purity and identity was confirmed using Western Blot as a part of the quality control testing. The representative SDS-PAGE and Western blot data is shown in the FIGS. 3A-3D and FIG. 4.

### Results:

The target cDNA sequence is shown in FIG. 1 and the resulting protein sequence is shown in FIG. 2. hrVCP according to these sequences was effectively expressed and confirmed with SDS-PAGE analysis as shown in FIGS. 3A-3D with purified hrVCP shown in FIG. 4.

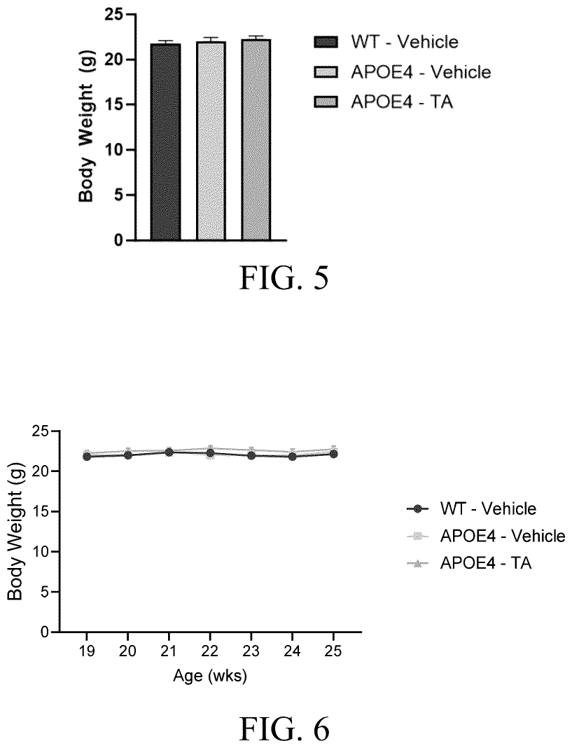

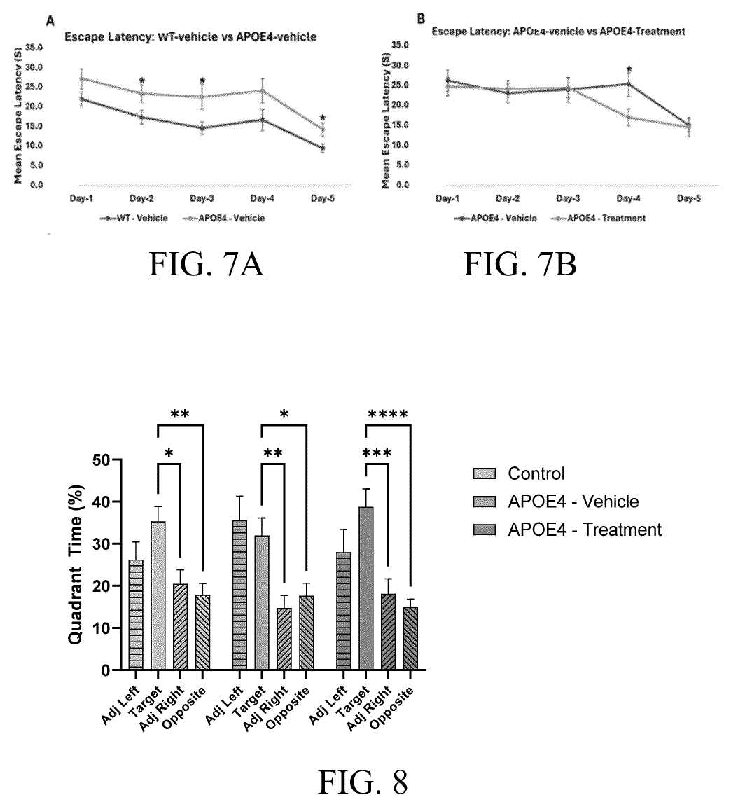

Upon analysis of the results of the foregoing experiments, it was observed that there were no significant differences in the body weights between the treatment groups prior to treatment and MWM study initiation (FIG. 5). Also, there was no significant difference between the groups until week 25 (FIG. 6). There was a difference in the spatial learning abilities of WT-vehicle and APOE4-vehicle groups on all 5 days and the difference was statistically significant on days 2, 3 and 5 as shown in FIGS. 7A-7D.

The mean escape latencies for APOE4-vehicle mice was on the higher side indicating that they took more time in finding the escape platform during the spatial learning paradigm of MWM.

The mice with better learning ability were expected to spend more time in the target quadrant as compared to other quadrants, particularly the opposite quadrant during the probe trial (FIG. 8). APOE4-vehicle mice spent more time in the adjacent left quadrant than that of the Target quadrant. Mice from WT-vehicle group spent more time in the Target quadrant (as compared to the other quadrants). However, mice from APOE4-vehicle group had spent more time in the adj left Q (35.56 Sec) as compared to the other quadrants (Target Q=31.97 Sec). hrVCP treatment outperformed vehicle treated mice. The hrVCP-treated mice spent more time in the target quadrants than the other quadrants with very significant to very highly significant differences between the time spent in the target Q vs opposite/Other Qs.

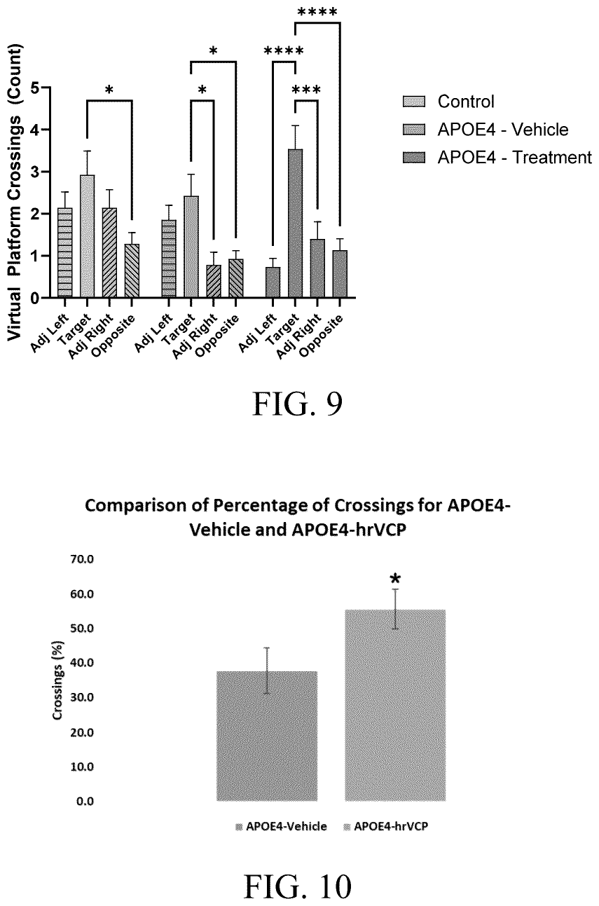

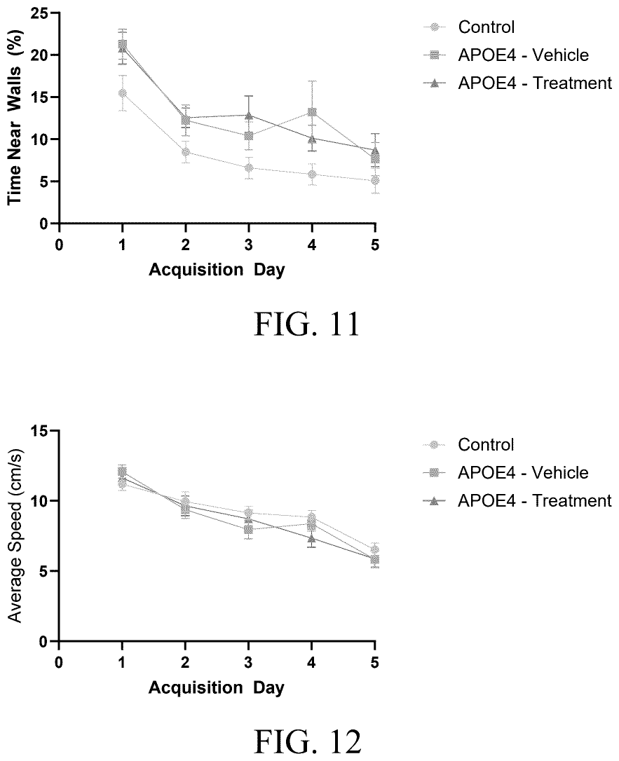

The data for the virtual crossings showed a similar trend, where hrVCP treated mice showed Two-way ANOVA found a significant effect of Quadrant, Treatment, and Treatment×Quadrant interaction (FIG. 9). Dunnett's post-hoc test found significantly lower virtual platform crossings in the opposite quadrant compared to target in the Control, APOE4-Vehicle, and APOE4-treatment groups. The number of crossings by hrVCP group in the Target quadrant was very highly significant (P<0.0001) as compared to the target quadrant (FIG. 9). hrVCP treatment improved the number of virtual crossings in the target quadrant significantly (FIG. 10). This clearly indicated better cognitive performance of hrVCP treatment group as compared to the vehicle treated group. There was no significant difference between different groups in terms of time spent near the walls (FIG. 11) and swimming speed (FIG. 12). This suggested that the observed differences in the mean escape latencies were not due to any effect on the motor function, but due to the cognitive improvement by hrVCP treatment in APOE4 mice.

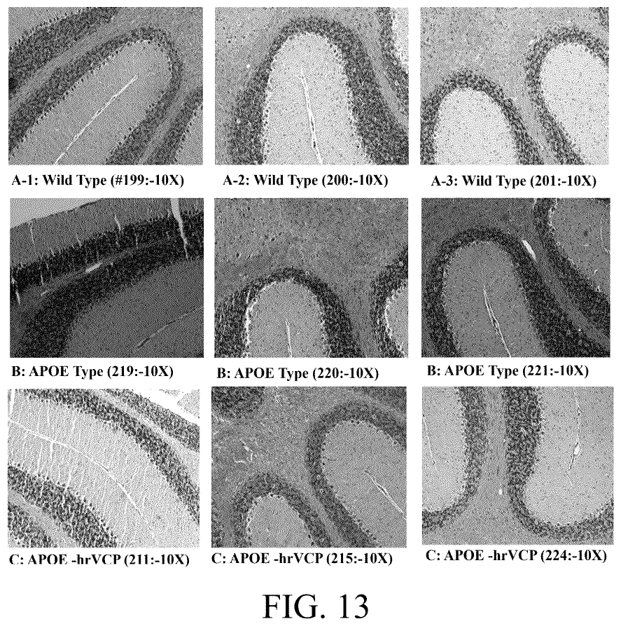

A representative hematoxylin and eosin (H&E) staining of the sagittal section (8 μm thick) of the right hemisphere of the brain including cerebellum from the WT-mice (A) and APOE4-vehicle (B) mice is shown in FIG. 13. The three critical cell layers of the cerebellum were observed, and the findings were as follows.

Data indicated that the molecular cell layer (ML) containing small, scattered basket cells and stellate cells were evident in both the representative slides from the control (Left) and the APOE4-vehicle groups.

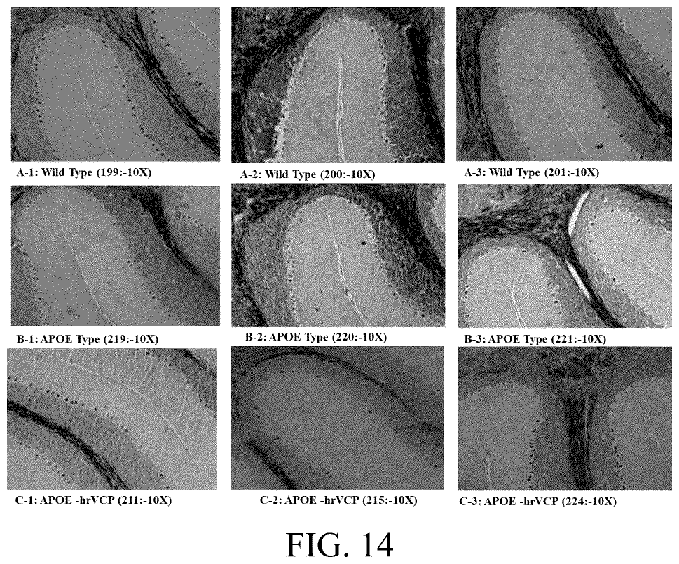

The representative brain sections from the cerebellum (same area used for H&E staining; FIG. 13) were stained using Bielschowsky silver stain (FIG. 14). The myelinated white matter of the cerebellar cortex stains dark grey using silver stain. As shown in A of the representative section from cerebellum of WT-vehicle mice, there was no visible plaque. Similarly, no plaque-like structures were observed in APOE4-vehicle KI mice (Panel B) and hrVCP treated mice (Panel C). Amyloid plaques were also not observed in any of the APOE4-vehicle or APOE4-hrVCP treated (APOE4-treatment) mice (Panels, B and C, respectively) investigated for histological observations (total APOE4 mice, n=6, n=3 shown here).

### Discussion

Transgenic Mice carrying human AD predisposing genes, are widely used as the most appropriate animal models for aging and cognitive studies due to their similarities with human immune responses. There are many well characterized knock-in (KI), knock out (KO) mice models of AD. Use of APOE ε4 mouse model in the early stages of development could help in early detection of microhemorrhage. APOE ε4 knock-in (HAbeta/APOE4/Trem2*R47H, LOAD2) mice supplied by the JAX laboratory were used for the current study. The mice were genotyped by the JAX laboratory before supplying the same. These mice have been developed for the late onset AD (LOAD), and thus the name LOAD2. As the name suggests, these mice have human APOE ε4 allele. The key features of these mice are mutations in TREM2 R47H, App gene and replacement/mutation of mouse Apo E gene with that of APOE ε4 gene resulting in AD pathology independent of amyloid and phosphorylated tau. These mice show the presence of insoluble Aβ (Aβ40 or Aβ42) and other features of LOAD as described by the JAX Laboratories. These mice can be used for Alzheimer's disease, lipoproteins, arteriosclerosis, and coronary heart disease. Also, these mice have been extensively characterized, and the data has been published. In that study, these mice have shown increased level of Aβ42 in the mice brain (particularly females; Aβ40 in males), and increased plasma neurofilament light chain (NfL) and neuronal loss in the cortex by 18 months of age. These mice have also shown impairment in the cognitive function in this study employing a touchscreen-based cognitive task. In conclusion, these mice form a model for preclinical studies for LOAD independent of amyloid plaques or phosphorylated tau pathology and were thus useful for the current study. VCP has been tested and shown to improve cognitive function in a Mo/Hu APPswe PS16E9 transgenic mouse model of amyloid pathology both after intracranial administration and after being administered intranasally. However, it was never tested in the LOAD model of AD where neurodegeneration is evident without amyloid and tau pathology. In AD, treatments are often via chronic administration of a drug using intracranial, or even intravenous (IV) bolus or infusion and requires intervention by skilled medical professionals. In the present study though, the intranasal route of administration was explored. This route of administration bypasses the blood brain barrier (BBB) and VCP has been shown to be capable of being delivered to the brain via this route of administration in a mouse model of AD. However, the concentration of the protein delivered may be in nanogram or picogram level. Thus, it was decided to use the humanized recombinant version of VCP (hrVCP) that has been shown to be several folds more potent than that of VCP and that inhibits the complement system at a very low concentration. This hrVCP was expressed using CHO cells as those cells were well characterized and many biologics for treating humans have been developed employing these cells. Ten microliters of hrVCP solution (approx. 1 mg/mL) was administered in each nostril in female APOE ε4 hAbeta/APOE4/Trem2*R47H, LOAD2 mice. The vehicle was administered to the transgenic control mice and to the wild type (WT) mice (C57BL/6J). As there was no data published on LOAD2 mice while performing these studies, it was important to first demonstrate that there was a difference between the wild type (WT) C57BL/6J that do not show LOAD pathology and APOE4-vehicle treated mice. Thus, pair-wise comparisons were made to evaluate any differences in the cognitive abilities of vehicle treated WT-vehicle mice and APOE4-vehicle mice, followed by APOE4-Treatment (or APOE4-hrVCP) groups. Morris water maze (MWM) was selected as a model for the assessment and comparison of the cognitive abilities, as this was a well-established model for studying learning and memory and many mouse models of AD have shown cognitive deficit at an early age using this model. The protocol described above in the materials and methods section was followed in characterization of various mice strains, and was the mice were independently assessed. As outlined in FIG. 5, there was no significant difference in the baseline body weights (BW) of the APOE4-vehicle treated mice (LOAD-2) and WT-vehicle treated mice before initiation of the study (Week-19). The trend was similar throughout the study duration (FIG. 6). The hrVCP treatment also did not show any effect on BW of mice after one month of treatment. No signs of irritation were reported during intranasal formulation. Thus, it can be concluded that hrVCP formulation was palatable and safe in mice.

It was important to consider the effect on BW in these mice as these are sensitive to the environmental changes in diet and high fat diet induces AD pathology in these mice. The data indicated that APOE ε4 mice (despite treatment/vehicle administration) were slightly on the higher side in terms of BW than that of WT, but the effect was not significant throughout the study and there was no significant change from the initial weight (FIGS. 5-6).

When the mice were subjected to spatial learning, there was a significant difference between WT-vehicle and APOE4-vehicle treated mice as shown in the FIG. 7A. The difference between the two groups were statistically significant on days 2, 3, and 5. This was also supported by the data for the distance travelled (cm) in these mice. Throughout spatial learning paradigm APOE4-vehicle treated mice have to travel longer distance as compared to the WT-vehicle treated mice to find the hidden escape platform. The mean escape latency data to locate the submerged platform data suggests that hrVCP treatment has showed improvement in the spatial learning as compared to that of the vehicle treated APOE4 group (FIGS. 7A-7B).

During the probe trial, which was designed to study the effect on the short-term memory loss typically observed in early stages of the disease in AD patients, the APOE4-hrVCP treatment group showed better performance than that of APOE4-vehicle treated group. The time spent by hrVCP treatment group in the target quadrant was statistically very highly significant as compared to the other quadrants, particularly in the opposite quadrant (P<0.0001). The effect was less pronounced in APOE4-vehicle group (FIG. 9).

Transgenic Mice carrying human AD predisposing genes, are widely used as most appropriate animal models for aging and cognitive studies due to their similarities with human immune responses. There are many well characterized knock-in (KI), knock out (KO) mice models of AD. Use of the APOE ε4 mouse model in the early stages of development could help in early detection of microhemorrhage. APOE ε4 knock-in (HAbeta/APOE4/Trem2*R47H, LOAD2) mice supplied by the JAX laboratory were used for the current study. The mice were genotyped by the JAX laboratory before supplying the same. These mice have been developed for the late onset AD (LOAD), and thus the name LOAD2. As the name suggests, these mice have a human APOE ε4 allele. The key features of these mice are mutations in TREM2 R47H, App gene and replacement/mutation of mouse ApoE gene with that of APOE ε4 gene resulting in AD pathology independent of amyloid and phosphorylated tau. Also, these mice have been extensively characterized, and the data has been published. In this study, these mice have shown increased level of Aβ42 in the mice brain (particularly females; Aβ40 in males), and increased plasma neurofilament light chain (NfL) and neuronal loss in the cortex by 18 months of age. These mice have also shown impairment in the cognitive function in this study employing a touchscreen-based cognitive task. In conclusion, these mice form a model for preclinical studies for LOAD independent of amyloid plaques or phosphorylated tau pathology and thus used for the current study. VCP has already been tested and shown to improve cognitive function in Mo/Hu APPswe PS16E9 transgenic mouse model of amyloid pathology both after intracranial administration and after being administered intranasally, but not hrVCP. However, it was never tested in the LOAD model of AD where neurodegeneration is evident without amyloid and tau pathology. hrVCP was tested for the first time using this LOAD2 model.

In AD, the treatment is chronic administration of drug using intracranial, or even intravenous (IV) bolus or infusion requires intervention by skilled medical professionals. Thus, the intranasal route of administration was explored in these experiments. This route of administration bypassed the blood brain barrier (BBB) and VCP has been shown to be delivered to the brain via this route of administration in a mouse model of AD. However, the concentration of the protein delivered may be in nanogram or picogram level. Thus, it was decided to use the humanized recombinant version of VCP (hrVCP) that has been shown to be several folds more potent than that of VCP and is expected to inhibit the complement system at a very low concentration. This was expressed using CHO cells as these are well characterized and many biologics for treating humans have been developed employing these cells. Ten microliters of hrVCP solution (approx. 1 mg/mL) was administered in each nostril in female APOE ε4 hAbeta/APOE4/Trem2*R47H, LOAD2 mice. The vehicle was administered to the transgenic control mice and to the wild type (WT) mice (C57BL/6J). As there was no data published on LOAD2 mice while performing these studies, it was important to first demonstrate that there was a difference between the wild type (WT) C57BL/6J that do not show LOAD pathology and APOE4-vehicle treated mice. Thus, pair-wise comparisons were made to evaluate any differences in the cognitive abilities of vehicle treated WT-vehicle mice and APOE4-vehicle mice, followed by APOE4-Treatment (or APOE4-hrVCP) groups.

Morris water maze (MWM) was selected as a model for the assessment and comparison of the cognitive abilities, as this was a well-established model for studying learning and memory and many mouse models of AD have shown cognitive deficit at an early age using this model. The protocol described in the materials and methods section were followed for the characterization of various mice strains, who were independently assess. As outlined in FIG. 5, there was no significant difference in the baseline body weights (BW) of the APOE4-vehicle treated mice (LOAD-2) and WT-vehicle treated mice before initiation of the study (Week—19). The trend was similar throughout the study duration (FIG. 6). The hrVCP treatment also did not show any effect on BW of mice after one month of treatment. No signs of irritation were reported during intranasal formulation. Thus, it was concluded that hrVCP formulation was palatable and safe in mice. It was important to consider the effect on BW in these mice as these are sensitive to the environmental changes in diet and high fat diet induces AD pathology in these mice. The present data indicated that APOE 4 mice (despite treatment/vehicle administration) were slightly on higher side in terms of BW than that of WT, but effect was not significant throughout the study and no significant change from the initial weight.

When the mice were subjected to spatial learning, there was a significant difference between WT-vehicle and APOE4-vehicle treated mice as shown in FIGS. 7A-7B. The differences between the two groups were statistically significant on days 2, 3, and 5. The mean escape latency data to locate the submerged platform data suggests that hrVCP treatment has showed improvement in the spatial learning as compared to that of the APOE4-vehicle group. During probe trial, which was designed to study the effect on the short-term memory loss typically observed in early stages of the disease in AD patients, APOE4-hrVCP treatment group showed better performance than that of APOE4-vehicle treated group. The time spent by hrVCP treatment group in the target quadrant was statistically very highly significant as compared to the other quadrants, particularly in the opposite quadrant (P<0.0001). The effect was less pronounced in APOE4-vehicle group. This group had spent more time in the opposite quadrant as compared to the APOE4-hrVCP group (FIG. 8). Similar behavior was observed for the number of crossings in the target quadrant, where hrVCP treatment group show more pronounced retention of memory as compared to the vehicle treatment groups (FIG. 9 and FIG. 10. The observed differences were only due to differences in cognitive capabilities and not due to motor or other behaviors as there is no difference between the groups in terms of time spent near the walls (FIG. 11) or average speed (FIG. 12). All these data attested to the ability of hrVCP in protection cognition associated with LOAD, independent of amyloid and tau pathology as evidenced by no plaques (FIG. 14). This study was performed at an early age in these mice (up to 25 weeks; approx. 6 months) and is believed to be the first preclinical study for therapeutic development in these mice.

After the MWM study, histology studies were performed using H&E staining and silver staining for any amyloid plaques. Other researchers have reviewed the APOE ε4 role in AD where they highlighted the effect of APOE ε4 mediated neuroinflammation in the white matter of the brain. It has been proposed that there is a link between APOE ε4 polymorphism and an increase of white matter hyperintensity lesioned areas observed by MRI in AD patients. White matter structural changes lead to cognitive dysfunction in AD patients. These changes could be attributed to APOE ε4 polymorphism leading to vascular dysfunction and could be further aggravated by the TREM2. In addition, the cells of cerebellum, particularly Purkinje cells are affected in AD even at an early age in a mouse model of AD (APPswe/PSEN16E9 mice) that was used by our group in the past. Thus, H&E staining of the different parts of the sagittal sections were performed including cerebellum. As shown in the FIG. 13, H&E staining showed all cell layers in WT-vehicle, APOE4-vehicle and APOE4-treatment groups. There was no plaque formation in these mice based on the silver staining data and as expected (FIG. 14).

It was believed that this was the first time that the cognitive abilities in these LOAD2 mice were compared to that of WT mice using MWM and that too as a part of the preclinical development. The mice used in this study were 6 months of age. hrVCP was administered intranasally and was unlikely to cause adverse effects similar to the antibodies targeting amyloid or complement as indicated by no changes in the body weight throughout the study.

### Conclusion

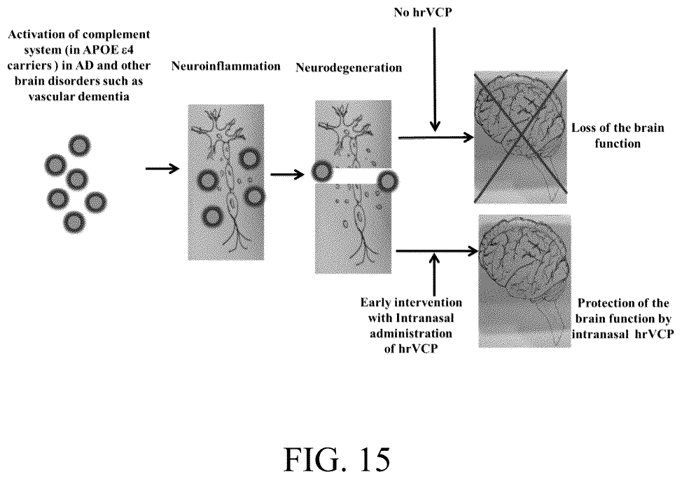

Based on the outcome of the current study, it was concluded that there was a difference in the spatial learning of WT and human APOE4 knock-in triple mutant (LOAD2) mice used for the current investigation. The hrVCP treatment improved cognitive function in LOAD2 mice at an early age for this study that was conducted for the first time. This widens and supports the indication of hrVCP to treat LOAD in addition to its role previously established in treating cognitive deficit in mice showing amyloid pathology. Thus, hrVCP can be administered intranasally to protect the cognitive function. ApoE ε4 allele carriers could encounter obstacles to longevity and the outcome of the current study indicated that hrVCP intervention can offer cognitive protection in APOE ε4 carriers. The outcome of the current study indicated that hrVCP intervention can offer cognitive protection in APOE ε4 carriers who develop symptoms independent of amyloid plaque formation and also for treating vascular dementia and other forms of dementia and brain disorders. In conclusion, the novel findings described herein can tremendously impact future therapeutic intervention by hrVCP for persons with APOE ε4. The finding of these studies are graphically represented in FIG. 15.

List of abbreviations: AD: Alzheimer's disease; ANOVA: Analysis of variance; CHO: Chinese hamster ovary; CNS: Central nervous system; DNA: Deoxyribose nucleic acid; FT: Flow through; hrVCP: humanized recombinant vaccinia virus complement control protein; MW: Molecular weight marker; NFT: Neurofibrillary tangle; ORF: Open reading frame; QC: Quality control; RM-ANOVA: Repeated Measures ANOVA; RNA: Ribose nucleic acid; rVCP: Recombinant vaccinia virus complement control protein; SDS-PAGE: Sodium dodecyl-sulfate polyacrylamide gel electrophoresis; VCP: Vaccinia virus complement control protein; SCI: Spinal cord injury; and TBI: Traumatic brain injury.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Hippius H, Neundörfer G. The discovery of Alzheimer's disease. Dialogues Clin Neurosci. 2003; 5.
2. Hunter P. The controversy around anti-amyloid antibodies for treating Alzheim.er's disease: The European Medical Agency's ruling against the latest anti-amyloid drugs highlights the ongoing debate about their safety and efficacy. EMBO reports.2024; 25.
3. Solopova E., Romero-Fernandez W, Harmsen H, Ventura-Antunes L, Wang E, Shostak A, et al. Fatal iatrogenic cerebral 0-amyloid-related arteritis in a woman treated with lecanemab for Alzheimer's disease. Nat Commun. 2023; 14.
4. Cummings J, Osse A M L, Cammann D, Powell J, Chen J. Anti-amyloid monoclonal antibodies for the treatment of Alzheimer's Disease. BioDrugs. 2024; 38.
5. Amelimojarad M, Amelimojarad M, Cui X. The emerging role of brain neuroinflammatory responses in Alzheimer's disease. Front Aging Neurosci. 2024; 16.
6. Leng F, Edison P. Neuroinflammation and microglial activation in Alzheimer disease: where do we go from here?. Nat Rev Neurol. 2012; 17.
7. Liu P, Wang Y, Sun Y, Peng G. Neuroinflammation as a Potential Therapeutic Target in Alzheimer's Disease. Clin Interv Aging. 2022; 17.
8. Kotwal G J. Complement-initiated neuroinflammation and its role in early-stage Alzheimer's disease. Commentary. Curr Alzheimer Res, 2011; 8.
9. Chen Y, Yu Y. Tau and neuroinflammation in Alzheimer's disease: interplay mechanisms and clinical translation. J Neuroinflammation. 2023; 20.
10. Zhang W, Xiao D, Mao Q, Xia H. Role of neuroinflammation in neurodegeneration development. Signal Transduct Target Ther. 2023; 8.
11. Cummings J L, Gonzalez M I, Pritchard M C, May P C, Toledo-Sherman L M, Harris G A et al. The therapeutic landscape of tauopathies: challenges and prospects. Alz Res Therapy 2023; 15.
12. Seo D, Holtzman D M. Current understanding of the Alzheimer's disease-associated microbiome and therapeutic strategies. Exp Mol Med. 2024; 56.
13. Kivipelto M., Mangialasche F, Ngandu T. Lifestyle interventions to prevent cognitive impairment, dementia and Alzheimer disease. Nat Rev Neurol 2018; 14. Available from: 10.1038/s41582-018-0070-3.
14. Daly J 4th, Kotwal G J. Pro-inflammatory complement activation by the A beta peptide of Alzheimer's disease is biologically significant and can be blocked by vaccinia virus complement control protein. Neurobiol Aging. 1998; 19.
15. Shi Q, Chowdhury S, Ma R, Le K X, Hong S, Caldarone B J, Stevens B, Lemere C A.

Complement C3 deficiency protects against neurodegeneration in aged plaque-rich APP/PS1 mice. Sci Transl Med. 2017; 9.
16. Shah A, Kishore U, Shastri A. Complement System in Alzheimer's Disease. Int J Mol Sci. 2001; 22.
17. Chernyaeva L, Ratti G, Teirila L, Fudo S, Rankka U, Pelkonen A, et al. Reduced binding of apoE4 to complement factor H promotes amyloid-O oligomerization and neuroinflammation. EMBO Rep. 2023; 24.
18. Eikelenboom P, Hack C E, Rozemuller J M, Stam F C. Complement activation in amyloid plaques in Alzheimer's dementia. Virchows Arch B Cell Pathol Incl Mol Pathol. 1989; 56.
19. Ng A N, Salter E W, Georgiou J, Bortolotto Z A, Collingridge G L. Amyloid-β1-42 oligomers enhance mGlu5R-dependent synaptic weakening via NMDAR activation and complement C5aR1 signaling. iScience. 2023; 26.
20. Shen Y, Lue L, Yang L, Roher A, Kuo Y, Strohmeyer R, et al. Complement activation by neurofibrillary tangles in Alzheimer's disease. Neurosci Lett. 2001; 305.
21. Gal J, Katsumata Y, Zhu H, Srinivasan S, Chen J, Johnson L A, et al. Apolipoprotein E Proteinopathy Is a Major Dementia-Associated Pathologic Biomarker in Individuals with or without the APOE Epsilon 4 Allele. Am J Pathol. 2022; 192.
22. Yin C, Ackermann S, Ma Z, Mohanta S K, Zhang C, Li Y et al. ApoE attenuates unresolvable inflammation by complex formation with activated Clq. Nat Med. 2019; 25.
23. Sun X Y, Yu X L, Zhu J, Li L J, Zhang L, Huang Y R, et al. Fc effector of anti-Aβ antibody induces synapse loss and cognitive deficits in Alzheimer's disease-like mouse model.

### Signal Transduct Target Ther. 2023; 8.

24. Troutwine B R, Hamid L, Lysaker C R, Strope T A, Wilkins H M. Apolipoprotein E and Alzheimer's disease. Acta Pharm Sin B. 2022; 12.
25. Qin Q, Wang M, Yin Y, Tang Y. The Specific Mechanism of TREM2 Regulation of Synaptic Clearance in Alzheimer's Disease. Front Immunol. 2022; 13.
26. Raber J, Huang Y, Ashford J W. ApoE genotype accounts for the vast majority of AD risk and AD pathology. Neurobiol Aging. 2004 May-June; 25(5):641-50.
27. Abondio P, Sazzini M, Garagnani P, Boattini A, Monti D, Franceschi C, Luiselli D, Giuliani C. The Genetic Variability of APOE in Different Human Populations and Its Implications for Longevity. Genes (Basel). 2019; 10.
28. Shen Y, Lue L, Yang L, Roher A, Kuo Y, Strohmeyer R, et al. Complement activation by neurofibrillary tangles in Alzheimer's disease. Neuroscience letters 2001; 305.
29. Hong S, Beja-Glasser V F, Nfonoyim B M, Frouin A, Li S, Ramakrishnan S, Merry K M, et al. Complement and microglia mediate early synapse loss in Alzheimer mouse models. Science. 2016; 352.
30. Lian H, Litvinchuk A, Chiang A C, Aithmitti N, Jankowsky J L, Zheng H. Astrocyte-Microglia Cross Talk through Complement Activation Modulates Amyloid Pathology in Mouse Models of Alzheimer's Disease. J Neurosci. 2016; 36.
31. Batista A F, Khan K A, Papavergi M T, Lemere C A. The Importance of Complement-Mediated Immune Signaling in Alzheimer's Disease Pathogenesis. Int J Mol Sci. 2024; 25.
32. Bermejo-Pareja F, Del Ser T. Controversial Past, Splendid Present, Unpredictable Future: A Brief Review of Alzheimer Disease History. J Clin Med. 2024; 13.
33. Zhang Y, Chen H, Li R, Sterling K, Song W. Amyloid j-based therapy for Alzheimer's disease: challenges, successes and future. Signal Transduct Target Ther. 2023; 8.
34. Rogers, M. B. Is ARIA an Inflammatory Reaction to Vascular Amyloid? Alzforum. 2023
35. Honig L S, Barakos J, Dhadda S, Kanekiyo M, Reyderman L, Irizarry M, et al. ARIA in patients treated with lecanemab (BAN2401) in a phase 2 study in early Alzheimer's disease. Alzheimers Dement (N Y). 2023; 9.
36. Foley K E, Wilcock D M. Three major effects of APOE ε4 on Aβ immunotherapy induced ARIA. Front Aging Neurosci. 2024; 16.
37. Kotwal G J and Moss B. Vaccinia virus encodes a secretory polypeptide structurally related to complement control proteins. Nature. 335, 176-178 (1988).
38. Kotwal G J, Isaacs S N, McKenzie R, Frank M M, Moss B. Inhibition of the complement cascade by the major secretory protein of vaccinia virus. Science. 1990; 250.
39. Isaacs S N, Kotwal G J, Moss B. Vaccinia virus complement-control protein prevents antibody-dependent complement-enhanced neutralization of infectivity and contributes to virulence. Proc Natl Acad Sci USA. 1992; 89. Online source:
40. Kulkarni A P, Govender D A, Kotwal G J, Kellaway L A. Modulation of anxiety behavior by intranasally administered vaccinia virus complement control protein and curcumin in a mouse model of Alzheimer's disease. Curr Alzheimer Res. 2011; 8.
41. Kulkarni A P, Pillay N S, Kellaway L A, Kotwal G J. Intracranial administration of vaccinia virus complement control protein in Mo/Hu APPswe PS1dE9 transgenic mice at an early age shows enhanced performance at a later age using a cheese board maze test. Biogerontology. 2008; 9.
42. Pillay N S, Kellaway L A, Kotwal G J. Early detection of memory deficits and memory improvement with vaccinia virus complement control protein in an Alzheimer's disease model. Behav Brain Res. 2008; 192.
43. Kulkarni A P, Govender D, Kellaway L A, Kotwal G J. Central nervous system distribution of the poxviral proteins after intranasal administration of proteins and titering of vaccinia virus in the brain after intracranial administration. Methods Mol Biol. 2012; 890.
44. Kotwal G J, Fernando N, Zhou J, Valter K. Exploring the potential benefits of vaccinia virus complement control protein in controlling complement activation in pathogenesis of the central nervous system diseases. Mol Immunol. 2014; 61.
45. Reynolds D N, Smith S A, Zhang Y P, Mengsheng Q, Lahiri D K, Morassutti D J, Shields C B, Kotwal G J. Vaccinia virus complement control protein reduces inflammation and improves spinal cord integrity following spinal cord injury. Ann N Y Acad Sci. 2004; 1035.
46. Hicks R R et al. Vaccinia virus complement control protein enhances functional recovery after traumatic brain injury. J Neurotrauma. 19, 705-714 (2002). Online source:
47. Anderson J B, Smith S A, Kotwal G J. Vaccinia virus complement control protein inhibits hyperacute xenorejection. Transplant Proc. 2002; 34. Online source: https://doi.org/10.1016/s0041-1345(02)02805-1.
48. Kahn D, Smith S A, Kotwal G J. Dose-dependent inhibition of complement in baboons by vaccinia virus complement control protein: implications in xenotransplantation. Transplant Proc. 2003; 35. Online source: https://doi.org/10.1016/s0041-1345(03)00485-8.
49. Ghebremariam Y T, Odunuga 00, Janse K, Kotwal G J. Humanized recombinant vaccinia virus complement control protein (hrVCP) with three amino acid changes, H98Y, E102K, and E120K creating an additional putative heparin binding site, is 100-fold more active than rVCP in blocking both classical and alternative complement pathways. Ann N Y Acad Sci. 2005; 1056.
50. Zhong M Z, Peng T, Duarte M L, Wang M, Cai D. Updates on mouse models of Alzheimer's disease. Mol Neurodegener. 2024; 19. Online source: Kotredes K P, Pandey R S, Persohn S, Elderidge K, Burton C P, Miner E W, et al. Characterizing molecular and synaptic signatures in mouse models of late-onset Alzheimer's disease independent of amyloid and tau pathology. Alzheimers Dement. 2024; 20.
51. Yokoyama M, Kobayashi H, Tatsumi L, Tomita T. Mouse Models of Alzheimer's Disease. Front Mol Neurosci. 2022; 15. Online source:
52. Fernández-Calle R, Konings S C, Frontiñán-Rubio J, Garciá-Revilla J, Camprubí-Ferrer L, Svensson M, et al. APOE in the bullseye of neurodegenerative diseases: impact of the APOE genotype in Alzheimer's disease pathology and brain diseases. Mol Neurodegeneration. 2022; 17. Available from: 10.1186/s13024-022-00566-4.
53. Mirza S S, Saeed U, Knight J, Ramirez J, Stuss D T, Keith J, Nestor S M, et al. Alzheimer's Disease Neuroimaging Initiative. APOE ε4, white matter hyperintensities, and cognition in Alzheimer and Lewy body dementia. Neurology. 2019; 93.
54. Mito R, Raffelt D, Dhollander T, Vaughan D N, Tournier J D, Salvado O, et al. Fibre-specific white matter reductions in Alzheimer's disease and mild cognitive impairment. Brain. 2018; 141.
55. McCorkindale A N, Mundell H D, Guennewig B, Sutherland G T. Vascular Dysfunction Is Central to Alzheimer's Disease Pathogenesis in APOE e4 Carriers. Int J Mol Sci. 2022; 23.
56. Chaudhari K, Wang L, Kruse J, Winters A, Sumien N, Shetty R, et al. Early loss of cerebellar Purkinje cells in human and a transgenic mouse model of Alzheimer's disease. Neurol Res. 2021; 43.
57. Zelek W M, Bevan R J, Morgan B P. Targeting terminal pathway reduces brain complement activation, amyloid load and synapse loss, and improves cognition in a mouse model of dementia. Brain Behav Immun. 2024; 118.
58. Kotwal G J. The Inheritance of a Normal Apo E Allele, Apo E3 or Apo E2 May Contribute to Diminished Incidence of Alzheimer's Disease Related Dementia and to Longevity. Austin Aging Res. 2017; 1.
59. Kotredes K P, et al. Characterizing molecular and synaptic signatures in mouse models of late-onset Alzheimer's disease independent of amyloid and tau pathology. Alzheimer's Dement. 2024; 20
60. Vijay Sharma and John H McNeill. To scale or not to scale: the principles of dose extrapolation. British Journal of Pharmacology. 2009; 157.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of reducing an APOE ε4 mediated cognitive deficit, comprising administering to a subject in need thereof an effective amount of a recombinant vaccinia virus complement control protein (hrVCP) polypeptide comprising a modified amino acid sequence including one or more amino acid substitutions to an amino acid sequence as set forth in SEQ ID NO: 2, wherein the one or more amino acid substitutions are selected from the group consisting of H98Y, E102K, E108K, E120K, and combinations thereof, provided at least one of the substitutions is E102K.

2. The method of claim 1, wherein the hrVCP polypeptide exhibits a complement activation regulatory activity greater than a complement activation regulatory activity of a polypeptide comprising the amino acid sequence as set forth in SEQ ID NO: 2.

3. The method of claim 1, wherein the subject is a human.

4. The method of claim 1, wherein the hrVCP polypeptide is expressed in a mammalian cell.

5. The method of claim 4, wherein the mammalian cell is a Chinese Hamster Ovary (CHO) cell.

6. The hrVCP polypeptide of claim 1, wherein the modified amino acid sequence comprises at least three of the amino acid substitutions selected from the group consisting of H98Y, E102K, E108K, E120K.

7. The method of claim 6, wherein the modified amino acid sequence comprises an amino acid sequence of SEQ ID NO: 3 or SEQ ID NO: 5.

8. The method of claim 2, wherein the complement activation regulatory activity comprises regulating activation of a classical complement activation pathway, an alternative complement activation pathway, or both the classical and alternative complement activation pathways.

9. The method of claim 1, wherein administering the hrVCP polypeptide comprises intrathecal, spinal, subdural, or intravenous administration.

10. The method of claim 1, wherein administering the hrVCP peptide comprises intranasal administration.

11. The method of claim 10, wherein administering the hrVCP polypeptide intranasally comprises intranasal administration using an inhalation device, a nasal spray, or a nasal tube.

12. The method of claim 1, wherein the subject has dementia.

13. The method of claim 12, wherein the subject has Alzheimer's disease or vascular dementia.

14. The method of claim 1, wherein the hrVCP is administered with a pharmaceutically-acceptable vehicle, carrier, or excipient.

15. The method of claim 1, wherein the hrVCP polypeptide is expressed in a mammalian or microbial cell using the sequence of SEQ ID NO: 4.

16. A method of treating dementia characterized by an APOE ε4 mediated cognitive deficit, comprising intranasally administering to a subject in need thereof an effective amount of a recombinant vaccinia virus complement control protein (hrVCP) polypeptide comprising a modified amino acid sequence including one or more amino acid substitutions to an amino acid sequence as set forth in SEQ ID NO: 2, wherein the one or more amino acid substitutions are selected from the group consisting of H98Y, E102K, E108K, E120K, and combinations thereof, provided at least one of the substitutions is E102K.

17. The method of claim 16, wherein the modified amino acid sequence comprises an amino acid sequence of SEQ ID NO: 3 or SEQ ID NO: 5.

18. The method of claim 16, wherein intranasally administering the hrVCP comprises intranasally administering the hrVCP using an inhalation device, a nasal spray, or a nasal tube.

19. A method of reducing complement activation mediated by APOE ε4 comprising administering to a subject in need thereof an effective amount of a recombinant vaccinia virus complement control protein (hrVCP) polypeptide comprising a modified amino acid sequence including one or more amino acid substitutions to an amino acid sequence as set forth in SEQ ID NO: 2, wherein the one or more amino acid substitutions are selected from the group consisting of H98Y, E102K, E108K, E120K, and combinations thereof, provided at least one of the substitutions is E102K.

20. The method of claim 19, wherein the complement activation is alone or in combination with human TREM2.

21. The method of claim 19, wherein the subject has a cognitive deficit secondary to aging, traumatic brain injury, or spinal cord injury.


SEQUENCE LISTING (5 sequences)

SEQ ID NO: 1 - DNA, 840 bp, Vaccinia virus
  source 1..840: mol_type = genomic DNA
tttttattatttgtacgatgtccaggataacatttttacggataaataaatatgaaggtggagagcgtgacgttcctgacattgttgggaataggatgcgttctatcatgctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgcctcgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttttataccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgc

SEQ ID NO: 2 - protein, 244 aa, Vaccinia virus
CCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 3 - protein, 244 aa, synthetic construct
CCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYYLIGKSKSYCELGSTGSMVWNPKAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 4 - DNA, 795 bp, synthetic construct
  source 1..795: mol_type = other DNA
atgtacagaatgcagctgctgtcctgcatcgccctgagcctggccctggtgaccaatagctgctgcacaatccccagcagacccatcaatatgaagttcaagaactccgtggagaccgacgccaacgccaactacaacatcggcgacacaatcgagtacctgtgcctgcctggctacaggaagcagaagatgggccctatctacgccaagtgcaccggcacaggctggacactgttcaatcagtgtatcaagagaagatgccctagccccagggatatcgataatggccagctggacatcggcggcgtggacttcggcagcagcatcacctactcctgcaatagcggctactacctgatcggcaagtccaagtcctactgtgagctgggctccaccggctccatggtgtggaaccctaaggcccccatctgtgagtccgtgaagtgtcagtcccccccttccatctccaacggcagacacaacggctacgaggacttctacaccgacggctccgtggtgacctactcctgtaactccggctacagcctgatcggcaactccggcgtgctgtgttccggcggcgagtggagcgatcctcccacctgccagatcgtgaagtgtcctcaccctaccatcagcaacggctacctgagctccggcttcaagaggtcctacagctacaacgataatgtggacttcaagtgcaagtacggctacaagctgtccggcagctcctccagcacatgctcccccggcaatacctggaagcccgagctgcccaagtgtgtgaggtga

SEQ ID NO: 5 - protein, 264 aa, synthetic construct
MYRMQLLSCIALSLALVTNSCCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYYLIGKSKSYCELGSTGSMVWNPKAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR